(19) United States
(12) Patent Application Publication
(10) Pub. No.: US 2025/0161363 A1
(43) Pub. Date: May 22, 2025

(54) COMPOSITIONS

(71) Applicant: UNIVERSIDAD DE CHILE, Santiago (CL)

(72) Inventors: Verónica Alejandra PALMA ALVARADO, Santiago (CL); Daniela Olivia CARRILLO ROJAS, Santiago (CL); Felipe Andrés OYARZÚN AMPUERO, Santiago (CL); Fernando Alfredo VALENZUELA AHUMADA, Santiago (CL); Maria Gabrela VILLAMIZAR SARMIENTO, Santiago (CL)

(21) Appl. No.: 18/840,593

(22) PCT Filed: Feb. 22, 2022
(86) PCT No.: PCT/IB2022/051550

§ 371 Date: Aug. 22, 2024

(51) Int. Cl. A61K 35/28; A61K 9/19; A61K 35/51; A61K 47/36; A61P 9/00; A61P 17/06; C12N 5/0775

(52) U.S. Cl. CPC A61K 35/28; A61K 9/19; A61K 35/51; A61K 47/36; A61P 9/00; A61P 17/06; C12N 5/0668

(57) ABSTRACT

The present invention relates to pharmaceutical compositions and methods for obtaining said pharmaceutical compositions comprising a conditioned culture medium of human mesenchymal stem cells and/or a combination of trophic factors expressed by human mesenchymal stem cells, and a polymeric matrix of hyaluronic acid. The present invention also relates to methods for preventing and/or treating patients having pathologies that generate tortuous vessels, or complications from the appearance of tortuous vessels, such as varicose veins, psoriasis, hemorrhoids, rosacea, acne, crystal skin, atopic dermatitis, among others.

### TECHNICAL FIELD

The present invention relates to the field of biomedicine and is related to providing pharmaceutical compositions related to skin conditions.

### BACKGROUND AND PRIOR ART

Angiogenesis is a natural process of formation of new blood vessels, in which endothelial cells migrate from pre-existing vessels and form new connections, to increase the vascular network at a given site. However, in adult individuals endothelial cells remain quiescent most of the time, activating against certain stimuli as part of an adaptive response to tissue hypoxia that occurs in a variety of situations such as wound healing, bone fracture repair, and the menstrual cycle.

The generation of new blood vessels is essential in the wound repair process because through the formation of new blood vessels the flow of nutrients and oxygen can be restored to the microenvironment in repair, where angiogenesis is the most relevant process. One of the key molecules within the angiogenesis process is vascular endothelial growth factor (VEGF).

Increased vascular permeability during angiogenic processes is associated with the absence of support cells along with the appearance of certain vascular structures called tortuous blood vessels. These types of blood vessels have a more curved morphology in relation to veins or arteries, which in most cases have a linear structure. Tortuous blood vessels have been widely described, and there is increasing evidence that these types of tortuous vessels are normally produced during angiogenic processes in different species to be later replaced by normal blood vessels. However, the incorrect regulation of pro-angiogenic and anti-angiogenic signals contributes in some cases to the permanent appearance of these signals. Its presence has been associated with certain pathologies such as retinopathies, cancer, atherosclerosis, hypertension, diabetes mellitus, and dermatosis, such as psoriasis.

Psoriasis mainly affects epithelial tissue by promoting altered release of pro-inflammatory cytokines such as IL-17, 22, 23 and TNF-α, generating chronic activation of innate and adaptive immune systems, adding to unregulated angiogenesis. Various environmental factors, such as infections, stress, medications, ultraviolet radiation, and skin injuries, can trigger or aggravate the disease that usually occurs after age 15, but can also manifest at a later age, around age 60, causing long-term damage in different organs and tissues. From the patient's point of view, it is a highly disabling disease because of its social, economic, and psychological repercussions that lead to discrimination on the part of others and isolation of the patient, which generates high rates of depression and anxiety.

For psoriasis, topical treatments are those used in a first line to fight plaque psoriasis when the body's surface is covered by up to 10% with these. The products available in the topical treatment market are: 1) salicylic acid ointment, 2) urea ointment, 3) salicylic acid oil, 4) salicylic acid sulfur ointment, 5) vegetable taries, 6) corticosteroid creams, ointments, or solutions and 7) vitamin D₃, topical calcineurin inhibitors (Kulawik-Pióro et al., 2021. Polymeric Gels and Their Application in the Treatment of Psoriasis Vulgaris: A Review. Int. J. Mol. Sci., 22(10), 5124).

Clobetasol, a corticosteroid, is the main drug used in the treatment of psoriasis vulgaris and represents the gold standard for the management of this disease. However, although conventional therapy is effective, it has a number of local and systemic adverse effects, such as atrophy and thinning of the skin, affecting its elasticity; in addition to the appearance of striae, rosacea, perioral dermatitis, acne, and purpura. On the other hand, systemic effects such as suppression of the hypothalamus-pituitary-adrenal axis, hyperglycemia and diabetes mellitus, mineralocorticoid effects, immunosuppressive effect, and toxicity in organs such as liver and kidney may also occur, affecting therapeutic adherence by the patient, and restricts its use to certain conditions in established schemes ( ). Another additional disadvantage in the use of this corticosteroid is inherent in its organoleptic characteristics, since it leaves a greasy sensation on the patient's skin, generating a number of inconveniences associated with its use, such as permanent stain of clothing and bedding. This set of adverse effects that generates the treatment with corticosteroids such as clobetasol, added to its organoleptic characteristics, directly affects the patient's therapeutic adherence, and therefore reaffirms the need to generate new technologies, as opposed to the treatment with this agent, to significantly minimize their adverse effects and therefore to restore the quality of life of patients.

Systemic treatments are used when topical therapy was not effective and when plaque formation is not localized in the joints of the body. Systemic therapy consists of immunosuppression of the patient, and the most commonly used agents are: 1) retinoids, 2) methotrexate, 3) hydroxyurea, 4) cyclosporine A, 5) fumaric acid, 6) antibiotics, 7) apremilast, and 8) biological drugs such as antibodies. Depending on the dose and duration of treatment, it can produce a number of side effects such as liver and kidney problems, such as inflammation and liver destruction, kidney damage, hypertension, or bone marrow immunosuppression (Kulawik-Pióro et al., 2021. Polymeric Gels and Their Application in the Treatment of Psoriasis Vulgaris: A Review. Int. J. Mol. Sci., 22(10), 5124).

In recent times, other types of alternative treatments to corticosteroids have emerged for psoriasis, which are called biological treatments. These types of biological drugs point to the blocking of specific receptors of key cellular components in the development of pathology, as well as the neutralization of cytokines secreted by these cells. Although patients with moderate to severe psoriasis may be treated with these medications, their prolonged use has certain limitations such as toxicity and intolerance. In addition, it may require administration in hospital facilities by specialized personnel, or at least maintain a cold chain that allows for the preservation of the injectable drug, and they have a high cost (between US$20,000-25,000 per year) (Kim et al., 2017. Diagnosis and management of psoriasis. Can. Fam. Physician., 63(4): 278-285). In this sense, despite the existing therapeutic alternatives, there is no treatment today that can keep patients permanently uninjured, preventing recurrent occurrences, or to achieve the disappearance of the pathology. This background represents an opportunity to look for new treatments that allow better results, with a better cost-benefit ratio than those currently available on the market.

Another disease that has been described in humans and related to the appearance of tortuous vessels is varices. This is a common condition that affects more than 25 million people in the United States alone. Varicose veins have a complex, multifactorial development that involves the interaction between genetic factors and predisposing risk factors including age, female sex, family history, pregnancy, obesity, and prolonged bipedal. Manifestations of this disease are usually found in the legs, but also in the scrotum (varicocele) and anus (hemorrhoids). These problems have been reported to affect approximately 30% of the world's population at some point in their lives, negatively impacting people's quality of life.

Treatment methods for varicose veins consist mainly of surgery in the most severe cases. But in most cases only a change in lifestyle is suggested as there is currently no effective method to prevent/eliminate varicose veins. In this way, it is clear that there is a need to find a biological alternative for the treatment of such conditions, or similar conditions.

An expert in the matter will understand that other similar conditions affecting the skin with an unregulated angiogenic component and that are susceptible to treatment with the present invention are: rosacea, acne, epidermolysis bullosa and atopic dermatitis.

Regenerative medicine is an interesting bet to address a possible therapeutic solution of psoriasis vulgaris based on the use of stem cells. Human mesenchymal stem cells (hMSC) are multipotent cells capable of self-renewal and differentiation in vitro in different types of tissues of the mesodermal lineage. hMSC are sources of trophic factors that modulate the immune system and induce stem cells present in tissues to repair damage, as well as other cells belonging to the immune system such as dendritic cells, T and B lymphocytes, and/or epithelial cells, endothelial cells, macrophages, among others. Various studies have revealed the therapeutic capacity of the hMSC based on the richness of its conditioned/secretome medium, which corresponds to the culture medium containing a mixture of biologically active and functional components that, when released into the medium, determine the cellular behavior.

Today, therapies are being sought to promote tissue repair and regeneration without the use of cells. This is why the use of secretome in relation to the administration of hMSC has numerous advantages, among them, avoiding tumorigenic potential, and faster supply. In addition, the use of secretome has a number of practical advantages, compared to the use of cells from the point of view of their industrial application, the following being: 1) easy storage (can be stored for long periods without the need for toxic cryopreservation and without loss of potency), 2) reduced legal requirements for use, 3) reduced risks of immune incompatibility, 4) reduced transmission of infections and 5) ease of handling.

An important point to note is that the safety, dose, and power of a product containing secretome can be evaluated in the same way that conventional biopharmaceutical products are evaluated, which, from a regulatory point of view, is essential to project the product to a national and international market (Teixeira et al., 2020. Mesenchymal stem cells secretome: Current trends and future challenges. Neural Regen. Res., 15(1), 75-77).

Wharton's gelatin corresponds to a connective tissue rich in collagen, hyaluronic acid, and sulfated proteoglycans in which the vascular structures of the umbilical cord are immersed, having, as their main function, to provide protection and support to them (Gupta y col., 2020, Umbilical cord-derived Wharton's jelly for regenerative medicine applications. J. Orthop. Surg. Res. 15:1-9). This gelatinous substance also contains hMSC inside called (hWJ-MSC). These cells are young, fibroblastoid in morphology, have low immunogenicity and also have no teratogenic potential (Marino et al., 2019. Mesenchymal stem cells from the Wharton's jelly of the human umbilical cord: Biological properties and therapeutic potential. Int. J. Stem Cell. 12:218-226). hWJ-MSC have been shown to have greater proliferative capacity, lifetime, and differentiation potential from other stem cell types such as bone marrow derivatives (BM MSC) and fat (AD MSC) since the expansion capacity of the latter decreases with age.

Although there are some studies that have evaluated the potential of secretome derived from hMSC derived from Wharton gelatin in some diseases, (Tansil Tan et al., 2020. New approach to deep diabetic foot ulcer (DFU) treatment-potential of secretome from Wharton's jelly mesenchymal stem cell therapy Int. J. Dermatol. Venereology Leprosy Sci., 3(2): 21-26), most studies describe intradermal administration of the acquired secretome and no topical administration of the secretome is described.

As part of regenerative medicine, biopolymer-based matrices have been developed to strengthen, replace, support organs, and repair tissues. The use of biopolymers has also been used to produce drug and/or drug release systems, without requiring the use of cells. In this respect, the use of hyaluronic acid (HA) has been used in both microneedle patches and sponges, both with potential biomedical applications for the transport of drugs.

Lyophilization is a process frequently used in the pharmaceutical and/or biomedical industry because it has a number of advantages from the point of view of storage and transport of lyophilized compositions. In addition, as mentioned above, the forms of presentation of the main topical products used in the treatment of psoriasis vulgaris are creams and ointments, which generate a number of problems in their acceptance by the patient due to the staining of their clothes, bedding and/or have bad smells. A lyophilized product such as the one described in this invention would eliminate or diminish these problems by reducing entry barriers of this product into the market.

No document of the state of the art before describes the surprising and synergistic effect of topically applying this secretome in combination with a polymeric agent such as hyaluronic acid, in a lyophilized form of presentation.

### BRIEF DESCRIPTION OF THE INVENTION

This invention describes a pharmaceutical composition that can be applied topically in patients who have tortuous vessels, or complications from the appearance of tortuous vessels, as is the case with psoriasis vulgaris, and in general it can be used in various dermal pathologies that have an immune or inflammatory component and/or vasculitis, such as rosacea, atopic dermatitis, scleroderma, bullous pemphigoid, lupus, among others. The pharmaceutical composition could even be applied in the treatment of infectious diseases or cancer. This pharmaceutical composition includes or comprises: (1) lyophilized conditioned culture medium of (e.g., human) mesenchymal stem cells, preferably human mesenchymal stem cells derived from Wharton's jelly and (2) a polymeric matrix, e.g., of hyaluronic acid.

The present invention also relates to a pharmaceutical composition comprising a combination of trophic factors expressed by human mesenchymal stem cells and a polymeric matrix of hyaluronic acid. The invention is also related to a method of producing the pharmaceutical compositions described herein. The pharmaceutical composition has properties for the regulation of angiogenesis, and which decreases the number of tortuous vessels in patients.

Embodiments of the invention are able to reduce and/or reverse the tortuosity of the vasculature which has not been possible with any of the current treatments for psoriasis. In addition, embodiments of the invention are able to reduce the diameter of one or more blood vessels, reduce the occurrence and/or severity of erythema, reduce epidermal thickness, reduce the number and/or severity of scales, spots, or lesions, reduce the area and/or severity or dry or cracked skin, and reduce occurrence and/or severity of itching, burning or discomfort. The solution is novel and seeks to open up in a market where the available treatments (corticosteroids such as clobetasol) do not generate high adherence in patients because their use is prolonged in time and/or cannot be easily scaled-up as they must be administered by medical specialists (biological therapies). On the other hand, the proposed solution would be administered in a simple way (a patch) that could be applied by the same patient.

### BRIEF DESCRIPTION OF FIGURES

FIG. 1: The angiogenic properties of hWJCM are maintained after the lyophilization process. FIG. 1A: Sponge HA hWJCM, as the final product of the freeze-drying process. Representative images obtained in different CAM of chicken embryos at day 12 of embryonic development (E12) after 4 days of stimulation with DMEM PRF (FIG. 1B), VEGF (FIG. 1C), hWJCM (FIG. 1D), HA Ctrl (FIG. 1E) and HA hWJCM (FIG. 1F), bar=5 mm are shown. FIG. 1G shows the quantification of the blood vessels obtained for each stimulus, normalized by the total number of vessels present in E8. One-way ANOVA with multiple comparisons, n=10, p<0.05. FIG. 1H: Measuring the diameter of the blood vessels surrounding the stimuli. DMEM PRF versus VEGF, hWJCM, HA Ctrl and HA hWJCM. Kolmogorov-Smirnov test, n=200, P<0.0001.

Differences between groups were considered non-significant when P≥0.05 (ns), significant when P<0.05 (*), highly significant when P<0.01 (**), and extremely significant when P<0.001 (***) and P<0.0001 (****).

FIG. 2: HA hWJCM treatment decreases the PASI index and induces recovery of body weight. The evolution of the characteristics of psoriatic lesions over a period of 11 days is shown: Erythema (FIG. 2A), scaling (FIG. 2B) and skin thickening (FIG. 2C) for each condition. In addition, the total PASI score for each experimental condition (FIG. 2D) is shown along with the percentage weight variation of the animals with respect to the first day of induction with PD (FIG. 2E), the treatments HA hWJCM, hWJCM and Clob in relation to the condition PD. Mann-Whitney test, n=3, P<0.05.

FIG. 3: Treatment of PD with HA hWJCM decreases the PASI index acutely by day 7 of treatment. FIG. 3A: The statistical analysis of the evolution of the PASI index (after starting treatments at day 4) at days 5, 7, 9 and 11 is shown. FIG. 3B: Corresponds to the statistical analysis of the dorsal skin thickness for the conditions studied at day 11. Mann-Whitney test P<0.05, n=3.

FIG. 4: The administration of treatments Clob, hWJCM, HA Ctrl and HA hWJCM decreases the clinical manifestations of PD. Representative photographs corresponding to skin morphological changes in the dorsal area of mice at day 13 are shown for the different groups under study. In Clob, hWJCM, HA Ctrl, HA hWJCM conditions, a decrease in the level of erythema, scales and skin thickness is observed with respect to the PD condition.

FIG. 5: Skin administration of HA hWJCM for 9 consecutive days improves the appearance of the back skin and decreases the thickness of the epidermis in a PD model. The conditions studied were Ctrl, PD, Clob, hWJCM, HA Ctrl and HA hWJCM.

FIG. 6: PD treatment with HA hWJCM does not reverse splenomegaly. The hepatic index is presented in FIG. 6A and the splenic index is presented in FIG. 6B for the different experimental conditions. Analysis performed on day 13 of the experimental protocol. One-way ANOVA, n=3, P<0.05.

FIG. 7: Treatment with HA hWJCM decreases both the number of normal and tortuous blood vessels and recovers the diameter of the vessels. Low- and high-resolution images (highlighted box) and representative skin sections are displayed on day 13 after receiving the indicated treatments. Compared to the Ctrl group, PD shows an increase in the number of vessels and also the presence of twisted spiral vessels. Bar=2.5 mm for low magnification images, Bar=0.5 mm for high magnification images.

FIG. 8: Treatment with HA hWJCM decreases both the number of normal and tortuous blood vessels and recovers the diameter of the vessels. FIG. 8A: The groups treated with Clob, hWJCM, HA Ctrl, and HA hWJCM show a decrease in the number of vessels compared to the PD condition. FIG. 8B: The tortuosity index is significantly reduced in groups treated with Clob, HA Ctrl, hWJCM, and HA hWJCM. Unidirectional ANOVA with repeated measurements: n=60. FIG. 8C: Only a decrease in the number of twisted blood vessels and hWJCM and HA hWJCM can be found. FIG. 8D: The complexity of tortuosity, expressed as the number of total segments per vessel, showed a reduction in HA hWJCM compared to the PD condition, being even different from hWJCM or HA Ctrl treatments. The complexity of tortuosity for vessels in Clob condition could not be obtained since they are extremely thin, in line with the literature. Unidirectional ANOVA with repeated measurements; n=30 for each group. FIG. 8E: Distribution graph for the diameter of blood vessels and their frequency in each condition. The hWJCM, HA Ctrl and HA hWJCM treatments can restore distribution to levels similar to Ctrl, the most significant difference being for HA hWJCM and hWJCM groups. Due to image resolution and excessive thinness of blood vessels, the diameter of blood vessels could not be established for the Clob condition. Kolmogorrov Smirnov test, α=0.05, n=60 for each measurement.

FIG. 9: Method of calculating tortuous blood vessels. Complexity of tortuous vessels was considered as the numbers of segments of a specific twisted vessel. This method was based on a published protocol used to quantify dendritic arborization (Uylings & van Pelt, 2002. Measures for quantifying dendritic arborizations. Network: Computation in Neural Systems, 13(3):397-414). As an example, a twisted blood vessel is highlighted being the segment number for that vessel 10 (S10). Tortuosity index (TI) was used for the quantification of tortuosity, calculated as the ratio of curve length (C) over the shortest line (L) connecting the starting and end point of a specific vessel. Created with BioRender.

### DETAILED DESCRIPTION OF THE INVENTION

As indicated above, this invention corresponds to a pharmaceutical composition comprising lyophilized conditioned culture medium of hMSCs, preferably hMSCs derived from Wharton jelly, or from the placenta, and a polymeric matrix, e.g., of hyaluronic acid. The invention also relates to a pharmaceutical composition comprising a combination of trophic factors expressed by human mesenchymal stem cells and a polymeric matrix of hyaluronic acid. The invention also relates to methods of producing the pharmaceutical compositions described herein.

In order to exemplify the invention and to show the main elements that make it up, the description of the invention shall be made, but such description shall not be considered as limiting, and as an expert in the matter shall understand, there are different modifications that can be made without varying the technical effects demonstrated throughout the present invention.

As used herein, the term ‘cell’ or ‘cells’ refers to mesenchymal stem cells (MSCs), preferably human mesenchymal stem cells (hMSCs). As defined by the International Society for Cellular Therapy (ISCT), the minimum criteria for a cell to be considered an MSCs is the expression of mesenchymal markers while lacking hematopoietic markers, along with the ability of the cell to differentiate into osteogenic, adipogenic, and chondrogenic lineages. Mesenchymal stem cells may be derived from dental tissue, skin, foreskin, salivary gland, limb buds, menstrual blood, placenta, bone-marrow (BM-MSCs), bone-marrow concentrate (BMC-MSCs), adipose tissue (AT-MSCs), or umbilical cord (including from umbilical cord blood (UCB-MSCs) or Wharton's jelly (WJ-MSCs)). In preferred embodiments, the MSCs are human MSCs. In more preferred embodiments, the MSCs are human MSCs derived from placenta or umbilical cord (i.e., hUCB-MSCs or hWJ-MSCs). In even more preferred embodiments, the MSCs are hWJ-MSCs. hWJ-MSCs have several advantages over other MSCs in that they retain characteristics of primitive stem cells, such as the expression of embryonic stem cell markers. Thus, hWJ-MSCs have a higher expression of pluripotency markers compared to MSCs from other sources.

As used herein, the Terms ‘Wharton's gelatine’, ‘Wharton's gelatin’, and ‘Wharton's jelly’ are used interchangeably to refer to a connective tissue originating from the umbilical cord, which connects the placenta and growing fetus during pregnancy. Therefore, mesenchymal stem cells from the umbilical cord provide a non-invasive source of cells, as the umbilical cord is considered medical waste, which is discarded after birth. In the umbilical cord, Wharton's jelly acts to resist torsional and compressive stresses imposed upon umbilical vessels during fetal development and comprises proteins, collagen, glycosaminoglycans, glycoproteins, mucopolysaccharides, cytokines, hyaluronic acid, chondroitin sulfate, and mesenchymal stem cells. Wharton's jelly may comprise up to 20% mesenchymal stem cells. Wharton's jelly may be extracted from the umbilical cord using methods that are standard in the art. Methods of obtaining Wharton's jelly from the umbilical cord may comprise, consist, or consist essentially of obtaining the umbilical cord, removing the external sheath, and removing the blood vessels.

MSCs can be obtained from a supplier, research facility or laboratory, or may be isolated from a tissue or sample using standard methods that are known in the art. Methods of isolating MSCs comprise a) obtaining a sample comprising MSCs, and b) isolating MSCs from the sample. Methods of isolating MSCs from a sample comprise, consist, or consist essentially of enzymatic methods or explant culture methods. hMSCs derived from the umbilical cord may be obtained from Sigma (C-12971, C-12972 or C-14091), Creative Bioarray (CSC-C2860), Caltag Medsystems (WJ-100-000), LifelineCellTech (FC-0020), ATCC (PCS-500-010), or AXOL (ax9003).

To isolate MSCs from a sample using explant culture methods, b) may comprise mechanically splitting the sample (i.e., by cutting or chopping) into pieces (i.e., around 0.1 to 20 mm, 0.1 to 10 mm, 0.5 to 20 mm, 0.5 to 10 mm, 0.5 to 8 mm, 0.5 to 7 mm, 0.5 to 5 mm, 1 to 10 mm, or 1 to 5 mm in length, width, depth, or diameter). In embodiments b) further comprises culturing the sample pieces. In embodiments, culturing the sample pieces allows the MSCs to grow out from the sample pieces onto the surface of a culture vessel. In embodiments the culturing is performed using a culture medium, such as a culture medium described herein. In embodiments b) further comprises passaging and culturing the MSCs using standard methods in the art, such as the methods described herein.

To isolate MSCs from a sample using enzymatic methods, b) may comprise incubating the sample with one or more enzymes. In embodiments one or more of the enzymes may degrade extra-cellular matrix. In embodiments, one or more of the enzymes may be selected from collagenase, trypsin, papain, dispase, accutase, hyaluronidase, elastase, pronase, or any commercial dissociation solution comprising one or more enzymes. After enzyme treatment, the MSCs may be cultured and passaged using methods that are standard in the art, such as the culture and passaging methods described herein.

Methods for isolating hUCB-MSCs comprise, consist, or consist essentially of: a) obtaining blood from an umbilical vein, preferably by venous puncture at the time of delivery, and b) isolating mononuclear cells from the blood sample. In embodiments, the method of isolating mononuclear cells is selected from: density gradient centrifugation (such as a Ficoll-Hypaque gradient), fluorescence activated cell sorting (FACS) and/or negative immunodepletion (i.e., of CD3+, CD14+, CD19+, CD38+, CD66b+, and glycophorin A+ cells, described in Lee et al., 2004. Isolation of multipotent mesenchymal stem cells from umbilical cord blood. Blood, 103(5): 1669-1675). In embodiments, a) further comprises storing the blood sample in the presence of an anticoagulant.

Methods for isolating hWJ-MSCs comprise, consist or consist essentially of a) obtaining an umbilical cord, b) cutting the umbilical cord into pieces (i.e., around 0.1 to 20 mm, 0.1 to 10 mm, 0.5 to 20 mm, 0.5 to 10 mm, 0.5 to 8 mm, 0.5 to 7 mm, 0.5 to 5 mm, 1 to 10 mm, or 1 to 5 mm in length, width, depth and/or diameter, or to a maximum of 10, 9, 8, 7, 6, 5, 4, 2, or 1 mm in length, width, depth and/or diameter), and c) isolating hWJ-MSCs from the umbilical cord. In embodiments, the umbilical cord of a) is a human umbilical cord. In embodiments, a) further comprises washing the obtained umbilical cord, preferably in a buffer solution such as phosphate-buffered saline. In embodiments, the method comprises removing blood vessels from the umbilical cord. In embodiments, the method further comprises a step between a) and b) of removing blood vessels from the umbilical cord. In embodiments the method further comprises a step between b) and c) of removing blood vessels from the umbilical cord. In embodiments c) comprises the use of either an enzymatic method or explant method of isolating hWJ-MSCs from the umbilical cord.

For enzymatic methods of isolating hWJ-MSCs from the umbilical cord, c) comprises incubating the umbilical cord with one or more enzymes. In embodiments, one or more of the enzymes digests the extracellular matrix. In embodiments the enzyme may be selected from: collagenase, trypsin, papain, accumax, dispase, accutase, hyaluronidase, elastase, pronase, or any commercial dissociation solution comprising one or more enzymes. In preferred embodiments, the one or more enzymes are a type I collagenase. In embodiments, the one or more enzymes is a collagenase is diluted in phosphate-buffered saline. In preferred embodiments, the incubation with one or more enzymes is performed with gentle agitation. In embodiments the incubation with one or more enzymes is performed at temperatures between 3° C. to 40° C., 3° C. to 39° C., 3° C. to 38° C., 10° C. to 40° C., 10° C. to 39° C., 10° C. to 38° C., 15° C. to 40° C., 15° C. to 39° C., 15° C. to 38° C., 20° C. to 40° C., 20° C. to 39° C., 20° C. to 38° C., 25° C. to 40° C., 25° C. to 39° C., 25° C. to 38° C., 30° C. to 40° C., 35° C. to 40° C., 35° C. to 39° C., 36° C. to 40° C., 36° C. to 39° C., or 36° C. to 38° C. In embodiments the incubation with one or more enzymes is performed for at least 5, 10, 15, 20, 25, 30, 35, 40, 45, 50, or 60 minutes. In embodiments the incubation with one or more enzymes is performed for at least 1, 2, 3, 4, 5, 6, 7, 8, 9, 10, 12, 13, 14, 15 or 16 hours. In embodiments, the incubation with one or more enzymes is performed between 0.1 to 72 hours, 0.1 to 48 hours, 0.1 to 36 hours, 0.1 to 24 hours, 0.1 to 20 hours, 0.1 to 18 hours, 0.5 to 72 hours, 0.5 to 48 hours, 0.5 to 36 hours, 0.5 to 24 hours, 0.5 to 20 hours, 0.5 to 18 hours, 1 to 72 hours, 1 to 48 hours, 1 to 36 hours, 1 to 24 hours, 1 to 20 hours, 1 to 18 hours, 2 to 72 hours, 2 to 48 hours, 2 to 36 hours, 2 to 24 hours, 2 to 20 hours, 2 to 18 hours, 5 to 72 hours, 5 to 48 hours, 5 to 36 hours, 5 to 24 hours, 5 to 20 hours, 5 to 18 hours, 10 to 72 hours, 10 to 48 hours, 10 to 36 hours, 10 to 24 hours, 10 to 20 hours, 12 to 20 hours, or 14 to 18 hours. In embodiments c) further comprises passing the incubation mixture (i.e., the umbilical cord with the one or more enzymes) through a filter, such as a nylon mesh filter. Preferably the nylon mesh filter has at least 0.1, 0.2, 0.5, 1, 2, 5, 10, 100 or 200 UM pores. In embodiments c) further comprises washing the incubation mixture with a culture medium or saline buffer (such as PBS). In embodiments, c) further comprises centrifuging the incubation mixture, preferably the filtered incubation mixture. In embodiments the centrifugation is performed for at least 1, 2, 5, 7 or 10 minutes, such as between 1 to 30 minutes, 1 to 20 minutes, 1 to 15 minutes, 5 to 30 minutes, 5 to 20 minutes, or 5 to 15 minutes. In embodiments the centrifugation is performed at least at 200, 500, 700, 1000, 1500 or 2000 rpm, such as between 200 to 10000 rpm, 200 to 8000 rpm, 200 to 5000 rpm, 200 to 4000 rpm, 200 to 3000 rpm, 500 to 10000 rpm, 500 to 8000 rpm, 500 to 5000 rpm, 500 to 4000 rpm, 500 to 3000 rpm, 1000 to 10000 rpm, 1000 to 5000 rpm, 1000 to 3000 rpm, or 1500 to 2500 rpm. In embodiments, c) further comprises suspending a cell pellet produced by centrifugation in cell culture medium. Examples of culture media are described herein. In embodiments, the method further comprises d) culturing the hWJ-MSCs. Examples of methods for culturing hWJ-MSCs are described herein. Methods for isolating hWJ-MSCs from Wharton's jelly using enzymatic methods are also described in U.S. Pat. No. 9,441,201, and Wang et al., 2008. Mesenchymal stem cells in the Wharton's jelly of human umbilical cord. Stem Cells, 22(7): 1330-1337, which are incorporated by reference.

For explant culture methods of isolating hWJ-MSCs from the umbilical cord, c) comprises isolating hWJ-MSCs using explant culture. In embodiments, the pieces of umbilical cord from b) are placed in culture vessels and cultured in a culture medium. Examples of culture media are described herein. Examples of culture methods are also described herein. In embodiments, c) further comprises removing the pieces of umbilical cord from the culture vessel after at least 1, 2, 3, 4, 5, 10, 15, or 20 days. In embodiments, the method further comprises d) culturing the hWJ-MSCs. Examples of methods for culturing hWJ-MSCs are described herein. In embodiments d) comprises culturing the hWJ-MSCs in a culture medium. Examples of culture media are described herein. Methods for isolating hWJ-MSCs from Wharton's jelly using explant methods are also described in Mennan et al., 2013. Isolation and characterization of mesenchymal stem cells from different regions of the human umbilical cord. BioMed Res. Int., ID 916136, incorporated herein by reference.

Methods of culturing MSCs are standard in the art and may comprise growing the MSCs in culture vessels in the presence of a culture medium. Examples of culture vessels are described herein. Examples of culture media are described herein. In embodiments the method further comprises passaging the MSCs, preferably when the MSCs reach between 50-100% confluency. Methods of passaging MSCs are described herein. In embodiments, the method further comprises changing the culture medium every 1 to 3 days, preferably at least every 2 or 3 days. In embodiments, the MSCs are cultured at a temperature between 35° C. to 40° C., preferably 35° C. to 39° C., more preferably between 36° C. to 38° C., even more preferably around 37° C. In embodiments, the method comprises culturing the MSCs in 2% to 10%, 2% to 9%, 2% to 8%, 2% to 7%, 3% to 7%, 3% to 6%, 4% to 7%, 4% to 6% or around 5% CO₂.

As used herein, the term ‘culture medium’, ‘culture media’ or ‘suitable culture medium’ refers to a solid, liquid, or semi-solid that supports the growth of MSCs, preferably hWJ-MSCs. For hWJ-MSCs, the culture medium may be selected from Dulbecco's modified Eagle's medium (DMEM), DMEM/F-12, α-minimum essential medium (α-MEM), StemPro® MSC SFM (Invitrogen), MesenCult™-XF Complete medium (Stem Cell Technologies), Mesencult® (Stem Cell Technologies), TheraPEAK™ MSCGM™ (Lonza, including BulletKit™ and Basal Medium), Mesenchymal stem cell growth medium 2 (PromoCell), Mesenchymal stem cell expansion medium (Sigma), Mesenchymal stem cell growth medium DXF (PromoCell), MSC NutriStem® XF medium (Sartorius), Human Mesenchymal-XF Expansion medium (Sigma), Stemline® Mesenchymal stem cell expansion medium (Sigma), StemXVivo® Media (R&D Systems), and/or mTeSR (Stem Cell Technologies). In embodiments the DMEM, DMEM/F-12 or α-MEM medium is supplemented with serum, preferably 0.1% to 30%, 1% to 30%, 1% to 25%, 1% to 20%, 1% to 15%, 5% to 20%, 5% to 15% or 8% to 12% serum in the medium (v/v). In preferred embodiments, the serum is fetal bovine serum (FBS) or human platelet lysate (such as PLTMax human platelet lysate (Sigma)). In embodiments, the medium is serum free. In embodiments, the medium further comprises additional cell culture reagents, such as cell culture reagents that are standard in the art. Examples of additional culture reagents include antibiotics (i.e., penicillin, streptomycin, neomycin, amphotericin B and/or gentamicin), proteins (such as Pepton™), glutamine (i.e., L-glutamine), pyruvic acid, glucose and/or commercial supplements (such as MesenCult™ MSC Stimulatory supplement).

A person skilled in the art appreciates that cells, including hMSCs, including placental MSCs, hUCB-MSCs and/or hWJ-MSCs may be cultured in any suitable culture vessel, including coated or un-coated culture vessels. In embodiments, the culture vessels may be coated in poly-lysine, poly-ornithine, collagen, gelatine, or commercial coatings such as MesenCult™-XF attachment substrate (STEMCELL Technologies), and/or Corning® CellBIND® (Sigma Aldrich). A person skilled in the art also appreciates that MSCs, including hWJ-MSCs, may alternatively be cultured using suspension cultures or 3D culture systems that are standard in the art and achieve the same invention as described herein.

As used herein, the term ‘passaging’, ‘splitting’, ‘subculturing’, ‘seeding’ or ‘planting’ refers to ‘splitting’ cell cultures to facilitate growth and expansion of the cells. Passaging may comprise, consist, or consist essentially of: a) incubating the MSCs with a dissociation solution, b) suspending the MSCs in a culture medium and c) transferring a percentage of the MSC suspension into one or more fresh (i.e., new or unused) culture vessels. In embodiments the dissociation solution of b) comprises, consists, or consists essentially of one or more enzymes selected from: collagenase, trypsin, papain, dispase, accutase, hyaluronidase, elastase, accumax, or pronase. In embodiments the dissociation solution may be any commercial dissociation solution such as the MesenCult™ Dissociation kit (STEMCELL Technologies). In embodiments a) is preceded by washing the MSCs, preferably with a buffer, such as a phosphate-buffered saline. In embodiments, a) is performed at temperatures between 35° C. to 40° C., preferably 35° C. to 39° C., more preferably between 36° C. to 38° C., even more preferably around 37° C. In embodiments, a) is performed for between 1 to 10 minutes, 1 to 9 minutes, 1 to 8 minutes, 1 to 7 minutes, 2 to 9 minutes, 2 to 8 minutes, 2 to 7 minutes, 2 to 6 minutes, 2 to 5 minutes, 2 to 4 minutes, 1 to 5 minutes, or 1 to 4 minutes. A person skilled in the art appreciates that the percentage of MSCs plated (also referred to as splitting ratio) c) can be adapted to suit the growth characteristics of the cells. Typical splitting ratios c) may be between 1:20, 1:15, 1:10, 1:7, 1:5 or 1:3. For example, a 1:3 split ratio demonstrates that, when plating the MSC suspension in the same size culture vessel, one third of the volume of MSC suspension (thus one third of the number of MSCs from the original culture vessel) is placed in the new culture vessel. If the MSCs are being placed into a larger culture vessel, the split ratio accounts for this. For example, if the MSCs are being plated into a culture vessel that is three times the size of the original culture vessel, a 1:3 split ratio would mean that all of the MSC suspension is transferred to the new culture vessel. In embodiments, passaging is performed on MSCs that have reached between 40% to 100%, 50% to 100%, 50% to 90%, 60% to 100%, 60% to 90%, 70% to 100%, or 70% to 90% confluency. In embodiments, the MSCs are passaged between 1 to 10 times, preferably 2 to 8 times.

As used herein, ‘confluency’ refers to the coverage of the surface of a culture vessel by MSCs. Confluency can be estimated by visualizing the MSCs under a standard light microscope.

The number of MSCs may be obtained using standard methods in the art. For example, the numbers of MSCs in a cell suspension (such as a cell suspension produced during passaging) may be counted manually (such as using a hemocytometer and light microscope) or using an automated process (such as coulter counters, flow cytometers or image-based counters). For manual cell counting, the cell density (cells/mL) is calculated as:

$$\frac{\begin{matrix}( average number of cells counter per square of hemocytometer \times \\ dilution factor )\end{matrix}}{volume of the square counted ( in mL )}$$

In this way, if it is required that a certain number of cells be plated (also referred to as ‘seeded’ or ‘planted’) in a culture vessel, the amount of cell suspension needed to provide the required number of cells to the new culture vessel can be calculated. The number of viable cells can also be counted manually or in an automated process by the addition of cellular dyes, such as Trypan Blue.

### Conditioned Culture Medium

As used herein, the term ‘conditioned culture medium’ or ‘secretome’ refers to culture medium that has been in contact with (i.e., in contact with or in culture with) MSCs. Conditioned culture medium therefore comprises agents secreted by, or released by, MSCs. In preferred embodiments, the conditioned culture medium comprises agents secreted by hMSCs, more preferably placental, hUCB-MSCs and/or hWJ-MSCs, even more preferably hWJ-MSCs. As used herein, the term ‘hWJCM’ refers to conditioned medium that has been in contact with hWJ-MSCs. In embodiments the conditioned culture medium comprises trophic factors secreted by the MSCs.

In embodiments, the conditioned culture medium comprises, consists or consists essentially of at least two, three, four, five, ten, fifteen, twenty, twenty-five, thirty, thirty-five, forty, forty-five, fifty or more trophic factors selected from: Angiogenin, Angiopoietin-1 (Ang-1), Angiopoietin-2 (Ang-2), amphiregulin, artemin, Coagulation factor 3, chemokine ligand 16 (CXCL16), dipeptidyl peptidase 4 (DPPIV), epidermal growth factor (EGF), endothelial growth factor (EG-VEGF), Endoglin, endothelin-1, acidic fibroblast growth factor (aFGF), basic fibroblast growth factor (bFGF), fibroblast growth factor 4 (FGF-4), fibroblast growth factor 7 (also referred to keratinocyte growth factor; FGF-7), glial cell derived neurotrophic factor (GDNF), Granulocyte-macrophage colony-stimulating factor (GM-CSF), Heparin-binding EGF-like growth factor (HB-EGF), hepatocyte growth factor (HGF), Insulin-like growth factor (IGF) binding protein 2 (IGFBP-2), Insulin-like growth factor (IGF) binding protein-3 (IGFBP-3), Interleukin 1 beta (IL-1B), transforming growth factor beta 1 (TGF-B1), LEPTIN, Monocyte chemoattractant protein-1 (MCP-1 also referred to as CCL2), Neutrophil collagenase (also known as matrix metalloproteinase-8, MMP-8), Matrix metallopeptidase 9 (MMP-9, also known as 92 kDa type IV collagenase, 92 kDa gelatinase or gelatinase B (GELB)), neuregulin beta 1 (NRG1-B1, also referred to as Heregulin beta 1), Platelet-derived endothelial cell growth factor (PD-ECGF), platelet-derived growth factor AA (PDGF-AA), platelet-derived growth factor AB (PDGF-AB), platelet-derived growth factor BB (PDGF-BB), PERSEFIN, Placental growth factor (PGF), Prolactin, Urokinase-type Plasminogen Activator (UPA), Vascular endothelial growth factor (VEGF), vascular endothelial growth factor C (VEGF-C), activin A, angiostatin, Hypoxia-inducible factor (HIF-1), Endostatin/CollagenXVIII, Insulin-like growth factor-1 (IGFBP-1), Macrophage Inflammatory Protein-1 alpha (MIP1α), Pentraxin-3 (PTX3), platelet factor 4 (PF4, also referred to as CXCL4), Maspin (Serpin B5), plasminogen activator inhibitor 1 (Serpin E1), Pigment epithelium-derived factor (PEDF, also referred to as Serpin F1), Tissue inhibitor matrix metalloproteinase 1 (TIMP-1), Metalloproteinase inhibitor 4 (TIMP-4), Thrombospondin 1 (TSP-1), Thrombospondin 2 (TSP-2), vasoinhibin, A disintegrin and metalloproteinase with thrombospondin motifs 1 (ADAMTS-1), and Netrins.

In embodiments, the conditioned culture medium comprises, consists or consists essentially of at least two, three, four, five, ten, fifteen, twenty, twenty-five, thirty, thirty-five or more of the following agents, e.g. in an aqueous solution: Angiostatin, activin A, HIF-1, Endostatin/CollagenXVIII, IGFBP-1, MIP1a, PTX3, PF4, Serpin B5, Serpin E1, Serpin F1, TIMP-1, TIMP-4, TSP-1, TSP-2, vasoinhibin, ADAMTS-1, HGF, Netrins, bFGF, Angiogenin, Ang-1, Ang-2, amphiregulin, artemin, Coagulation factor 3, CXCL16, DPPIV, EGF, EG-VEGF, Endoglin, endothelin-1, aFGF, FGF-4, FGF-7, GDNF, GM-CSF, HB-EGF, IGFBP-2, IGFBP-3, IL-1B, TGF-B1, LEPTIN, MCP-1, MMP-8, MMP-9, NRG1-B1, PD-ECGF, PDGF-AA, PDGF-AB, PDGF-BB, PERSEFIN, PGF, Prolactin, UPA, VEGF, and VEGF-C.

In embodiments, the conditioned culture medium comprises, consists or consists essentially of at least two, three, four, five, ten, fifteen or more of: Angiogenin, Ang-1, Ang-2, amphiregulin, artemin, Coagulation factor 3, CXCL16, DPPIV, EGF, EG-VEGF, Endoglin, endothelin-1, aFGF, bFGF, FGF-4, FGF-7, GDNF, GM-CSF, HB-EGF, HGF, IGFBP-2, IGFBP-3, IL-1B, TGF-B1, LEPTIN, MCP-1, MMP-8, MMP-9, NRG1-B1, PD-ECGF, PDGF-AA, PDGF-AB, PDGF-BB, PERSEFIN, PGF, Prolactin, UPA, VEGF, VEGF-C, HIF-1, activin A and angiostatin.

In embodiments the conditioned culture medium comprises, consists or consists essentially of at least two, three, four, five, ten, fifteen or more of: Angiostatin, Endostatin/CollagenXVIII, IGFBP-1, MIP1a, PTX3, PF4, Serpin B5, Serpin E1, Serpin F1, TIMP-1, TIMP-4, TSP-1, TSP-2, vasoinhibin, ADAMTS-1, HGF, Netrins and bFGF.

In embodiments, the conditioned culture medium comprises, consists or consists essentially of the following agents, e.g. in an aqueous solution: Angiogenin, Ang-1, Ang-2, amphiregulin, artemin, Coagulation factor 3, CXCL16, DPPIV, EGF, EG-VEGF, Endoglin, endothelin-1, aFGF, bFGF, FGF-4, FGF-7, GDNF, GM-CSF, HB-EGF, HGF, IGFBP-2, IGFBP-3, IL-1B, TGF-B1, LEPTIN, MCP-1, MMP-8, MMP-9, NRG1-B1, PD-ECGF, PDGF-AA, PDGF-AB, PDGF-BB, PERSEFIN, PGF, Prolactin, UPA, VEGF, VEGF-C, HIF-1, activin A and/or angiostatin.

In embodiments, the conditioned culture medium comprises, consists, or consists essentially of the following agents, e.g., in an aqueous solution: Angiostatin, Endostatin/CollagenXVIII, IGFBP-1, MIP1a, PTX3, PF4, Serpin B5, Serpin E1, Serpin F1, TIMP-1, TIMP-4, TSP-1, TSP-2, vasoinhibin, ADAMTS-1, HGF, Netrins and/or bFGF.

In embodiments the conditioned culture medium and/or the composition may also comprise cytokines such as interleukin 1 receptor antagonist (IL-1RA).

In embodiments, the trophic factors are expressed by the MSCs. However, a person skilled in the art appreciates that an artificial conditioned culture medium may be produced by substituting a suitable culture medium, that has not been exposed to MSCs, with at least two, three, four, five, ten, fifteen, twenty, twenty-five, thirty, thirty-five, forty, forty-five, fifty or more trophic factors described herein.

Methods of producing a conditioned culture medium or secretome are also described herein. In embodiments, the culture medium is produced by a method comprising: a) culturing MSCs in a suitable culture medium, and b) collecting conditioned medium therefrom. In embodiments, the method comprises an additional step before a) of isolating the MSCs from a sample. Examples of methods of isolating MSCs from a sample are described herein. In embodiments, a) further comprises passaging the MSCs. In embodiments, during the passaging step, the cell numbers are counted and 100 to 1,000,000 MSCs, preferably 1000 to 500,000 MSCs, more preferably, 1000 to 250,000 MSCs, or about 170,000 MSCs are plated into a new culture vessel. Methods for passaging and counting cells are described herein. In embodiments, a) further comprises culturing the MSCs (i.e., after passaging) until the MSCs reach between 40% and 100% confluency, preferably between 40% and 90%, more preferably between 50% and 80%, even more preferably between 60% and 90%. In embodiments, a) further comprises replacing the culture medium with fresh culture medium. Examples of suitable culture media described herein. In preferred embodiments, the culture medium is a serum free and/or phenol red-free medium. In embodiments, when the MSCs reach the desired confluency in a), the culture medium is replaced with fresh culture medium. In embodiments, the MSCs are cultured in the culture medium for between 2 to 100 hours, 2 to 72 hours, 5 to 100 hours, 5 to 72 hours, 2 to 60 hours, 5 to 60 hours, 2 to 50 hours, 5 to 50 hours, 10 to 100 hours, 10 to 72 hours, 10 to 60 hours, 10 to 50 hours, 15 to 100 hours, 15 to 72 hours, 15 to 60 hours, 15 to 50 hours, 20 to 100 hours, 20 to 72 hours, 20 to 60 hours, 20 to 50 hours, 24 to 72 hours, 24 to 60 hours, 24 to 50 hours, 24 to 48 hours, 48 to 72 hours, 30 to 70 hours, 38 to 58 hours, 40 to 56 hours, or 40 to 50 hours. In embodiments, step b) comprises transferring the culture medium from the cell culture vessel into a suitable container, such as a sterile container. In embodiments, the conditioned culture medium can be stored at temperatures between 10° C. to −130° C., preferably 4° C. to −120° C., more preferably −20° C. to −120° C., even more preferably between −80° C. to −120° C. In embodiments, the conditioned culture medium is lyophilized. Examples of lyophilization methods are described herein.

The present disclosure also provides ‘conditioned culture medium’ or ‘secretome’ obtained or obtainable by the method described herein.

### Composition

As used herein, the terms ‘composition’ or ‘pharmaceutical composition’ are used interchangeably and refer to any composition comprising, consisting, or consisting essentially of a conditioned culture medium described herein. In embodiments, the conditioned culture medium is lyophilized. In preferred embodiments, the composition further comprises, consists, or consists essentially of hyaluronic acid. In embodiments the hyaluronic acid is also lyophilized. In embodiments the polymer matrix is made by repetitive units of hyaluronic acid.

Hyaluronic acid (HA) is a linear polysaccharide composing of a repeated disaccharide unit of β-1,4 linked D-glucuronic acid (GlcA) and β-1,3 linked N-acetyl-D-glucosamine (GlcNAc). The polymer can then assemble into three-dimensional structures, referred to as ‘coils’ (or a ‘polymeric matrix’). As such the term ‘polymeric matrix’ as used herein refers to three-dimensional structures of hyaluronic acid. In embodiments, the coils in turn assemble to form spheroidal particles. In embodiments, hyaluronic acid may be lyophilized to produce a ‘sponge’, i.e., dried hyaluronic acid such that the spheroidal particles are able to absorb and release water-soluble molecules. In the liquid or lyophilized hyaluronic acid, the space between the HA spheroidal particles may be occupied by large-molecular weight, soluble HA whilst small-molecular weight, soluble HA may occupy inside and around the HA particles (Hadler et al., 1982. Ultrastructure of a hyaluronic acid matrix. PNAS, 79:307-309; and Mahedia et al., 2016. Clinical evaluation of hyaluronic acid sponge with zinc versus placebo for scar reduction after breast surgery. Plast Reconstr Surg Glob Open, 4(7): e791). In preferred embodiments, the sponge may further comprise, consist, or consist essentially of the lyophilized conditioned culture medium described herein, preferably the trophic factors produced by MSCs, as described herein. In embodiments, the sponge further comprises at least one, two, three, four, five, ten, fifteen, twenty, thirty or more trophic factors as described herein. In embodiments, the trophic factors are distributed in the space around and filling the HA particles. In embodiments, the sponge, when applied to the skin, releases water and the biologically active molecules. In embodiments, the sponge may be reconstituted before use in a liquid, such as water.

In embodiments, the concentration of hyaluronic acid in the composition before lyophilization is between 0.5 mg/mL to 100 mg/mL, 0.5 mg/mL to 90 mg/mL, 0.5 mg/mL to 80 mg/mL, 0.5 mg/mL to 70 mg/mL, 0.5 mg/mL to 60 mg/mL, 0.5 mg/mL to 50 mg/mL, 0.5 mg/mL to 40 mg/mL, 0.5 mg/mL to 30 mg/mL, 0.5 mg/mL to 25 mg/mL, 0.5 mg/mL to 20 mg/mL, 0.5 mg/mL to 15 mg/mL, 0.5 mg/mL to 10 mg/mL, 0.5 mg/mL to 5 mg/mL, 1 mg/mL to 100 mg/mL, 1 mg/mL to 80 mg/mL, 1 mg/mL to 60 mg/mL, 1 mg/mL to 50 mg/mL, 1 mg/mL to 40 mg/mL, 1 mg/mL to 30 mg/mL, 1 mg/mL to 25 mg/mL, 1 mg/mL to 20 mg/mL, 1 mg/mL to 15 mg/mL, 1 mg/mL to 10 mg/mL, 1 mg/mL to 5 mg/mL, 1 mg/mL to 4 mg/mL, 1 mg/mL to 3 mg/mL, 0.5 mg/mL to 3 mg/mL or 1 mg/mL to 2 mg/mL.

In embodiments, the content of hyaluronic acid present in a lyophilized pharmaceutical composition is between 0.5 mg/g to 100 mg/g, 0.5 mg/g to 90 mg/g, 0.5 mg/g to 80 mg/g, 0.5 mg/g to 70 mg/g, 0.5 mg/g to 60 mg/g, 0.5 mg/g to 50 mg/g, 0.5 mg/g to 40 mg/g, 0.5 mg/g to 30 mg/g, 0.5 mg/g to 25 mg/g, 0.5 mg/g to 20 mg/g, 0.5 mg/g to 15 mg/g, 0.5 mg/g to 10 mg/g, 0.5 mg/g to 5 mg/g, 1 mg/g to 100 mg/g, 1 mg/g to 80 mg/g, 1 mg/g to 60 mg/g, 1 mg/g to 50 mg/g, 1 mg/g to 40 mg/g, 1 mg/g to 30 mg/g, 1 mg/g to 25 mg/g, 1 mg/g to 20 mg/g, 1 mg/g to 15 mg/g, 1 mg/g to 10 mg/g, 1 mg/g to 5 mg/g, 1 mg/g to 4 mg/g, 1 mg/g to 3 mg/g, 0.5 mg/g to 3 mg/g or 1 mg/g to 2 mg/g.

In embodiments, the composition comprises at least 5%, 10%, 15%, or 20% (v/v) of conditioned culture medium in the total composition volume. In embodiments, the composition comprises at least 5%, 10%, 15%, or 20% (v/v) of conditioned culture medium in hyaluronic acid.

The composition may further comprise additional agents that are used for treating or preventing skin disorders/disease/conditions and/or diseases, disorders or conditions associated with tortuous blood vessels. In embodiments, the composition may further comprise anti-inflammatory agents including steroidal and non-steroidal drugs (including salicylic acid), antibiotics (such as doxycycline, metronidazole, minocycline, erythromycin, clindamycin), antimicrobials, brimonidine, antipruritics (such as clemastine, cyproheptadine, loratadine, hydroxyzine, ketotifen, promethazine, chlorpheniramine, alimemazine, pheniramine, nalmefene, doxepin, crotamiton), corticosteroids (such as hydrocortisone, triamcinolone and clobetasol), local anaesthetics (such as lidocaine, pramoxine or benzocaine), antipsoriatic agents, green tea, vitamin D analogues (including calcipotriene, calcitriol), collagen, chitosan, niacinamide, feverfew, calcineurin inhibitors (such as tacrolimus and pimecrolimus), benzoyl peroxide, moisturising agents, glycolic acid, witch hazel, essential oils, cetearyl alcohol, azelaic acid, and/or ivermectin.

Compositions of the present invention can have a selected viscosity by the addition of further reagents, such as water, glycerin, alcohol, oil, a silicone-containing compound, wax, and thickening agents. Compositions of the present invention may be produced as an emulsion (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydroalcoholic), anhydrous bases, gels, masks, ointment, or sponge.

As used herein, the terms ‘administering’, ‘administer’ or ‘administration’ means providing to a subject the composition described herein using any method of delivery known to those skilled in the art to treat or prevent a disease, disorder, or condition in a patient, particularly a skin disease, skin disorder or skin condition, and/or a disease, disorder or condition associated with tortuous blood vessels. Preferred routes or methods of delivery for the composition described herein include topical administration (including patches, microneedle patches, powder, liquid, gel, sponge, or cream formulations), or administration via subcutaneous or intradermal injection.

As used herein, the term ‘therapeutically effective amount’, ‘therapeutically effective dose’, ‘pharmaceutically effective amount’ or ‘pharmaceutically effective dose’ refers to an amount of the composition and/or trophic factors described herein that, when administered to a patient or subject with a disease, disorder or condition described herein is sufficient to cause a qualitative or quantitative reduction in the actual or predicted severity or frequency of symptoms of that disease, disorder or condition, a reduction of the actual or predicted pathology and/or delaying or preventing the actual or predicted onset of pathology or symptoms. In embodiments, the therapeutically effective amount of the composition and/or trophic factors described herein reduces the number of, or severity of, tortuous blood vessels in the subject. In embodiments, the therapeutically effective amount of the composition and/or trophic factors described herein reduces the blood vessel diameter in the skin of a subject. In embodiments, the therapeutically effective amount of the composition and/or trophic factors described herein reduces the symptoms or signs of the skin disease, disorder, or condition in the patient, such as a reduction in the degree of erythema, epidermal thickness, scales, spots, dry or cracked skin, itching, burning, lesions, or discomfort.

The composition as described herein may be administered only once. Preferably, the composition is administered multiple times. Certain factors may influence the dosage, application schedule, or concentration required to effectively treat a subject, including but not limited to the severity of the disease, disorder, or condition, previous or concurrent treatments, the general health and/or age of the subject, and other diseases present. It will also be appreciated that the effective dosage of the composition for treatment may need to increase or decrease over the course of a particular treatment.

In embodiments, the composition may be administered as an adjunct to, or as part of combination therapy with, other therapies that are used for treating or preventing skin disorders, diseases, or conditions, and/or diseases, disorders or conditions associated with tortuous blood vessels. In embodiments, the other therapy may comprise, consist or consist essentially of anti-inflammatory agents including steroidal and non-steroidal drugs (including salicylic acid), antibiotics (such as doxycycline, metronidazole, minocycline, erythromycin, clindamycin), antimicrobials, brimonidine, antipruritics (such as clemastine, cyproheptadine, loratadine, hydroxyzine, ketotifen, promethazine, chlorpheniramine, alimemazine, pheniramine, nalmefene, doxepin, crotamiton), corticosteroids (such as hydrocortisone, triamcinolone and clobetasol), local anaesthetics (such as lidocaine, pramoxine or benzocaine), antipsoriatic agents, green tea, vitamin D analogues (including calcipotriene, calcitriol), niacinamide, feverfew, calcineurin inhibitors (such as tacrolimus and pimecrolimus), benzoyl peroxide, moisturising agents, glycolic acid, witch hazel, essential oils, cetearyl alcohol, azelaic acid, and/or ivermectin.

In embodiments, the composition described herein may be used in the manufacture of a medicinal product and/or medicament for preventing and/or treating a disease, disorder, or condition, preferably a skin disease, disorder, or condition, and/or a disease, disorder or condition associated with tortuous blood vessels.

In embodiments, the compositions described herein are for use in the treatment or prevention of any of the diseases, disorder or conditions described herein.

In embodiments, there is provided a method of treating any of the diseases, disorders or conditions described herein. The method of treatment may comprise, consist, or consist essentially of administering to a subject the composition described herein, preferably a pharmaceutically effective amount of the composition.

As used herein, ‘lyophilization’ or ‘lyophilizing’ (also referred to in the art as ‘freeze-drying’ or ‘cryodessicating’) refers to the process of low temperature dehydration of (i.e., removal of water from) a product. Lyophilization methods are standard in the art and are described in Rey and May 2010. Freeze drying/lyophilization of pharmaceutical and biological products (3rd Edition), Drugs and the Pharmaceutical Sciences, 206, Costantino and Pikal, 2004. Lyophilization of biopharmaceuticals. American Association of Pharmaceutical Scientists, and Bhambere et al., 2015. Lyophilization/freeze drying—a review. World Journal of Pharmaceutical Research, 4(8): 516-543, which are incorporated by reference in their entirety). The method of lyophilization comprises, consist, or consist essentially of freezing the product (‘freezing’), and dehydrating the product by heating the product at low pressure (referred to herein as ‘primary drying’). Heating the product at low pressure allows sublimation of ice produced by the freezing step. In embodiments the freezing step may be performed in liquid nitrogen, a freezer (such as a cryogenic or mechanical freezer), a chilled bath (shell freezer), or in a freeze dryer.

In embodiments the freezing step of lyophilization is performed at a temperature below at least −10° C., −11° C., −12° C., −13° C., −14° C., −15° C., −16° C., −17° C., −18° C., −19° C. −20° C., −21° C., −22° C., −25° C., −30° C., −35° C., −40° C., −45° C., −50° C., −55° C. or −60° C. In embodiments the freezing step is performed over at least 1, 2, 3, 4, 5, 7, 8, 10, 12, 15, 17, 18, 20, 22, or 24 hours. In embodiments the freezing step may be performed at 800 to 1500 mbar, 850 mbar to 1100 mbar, 870 mbar to 1084 mbar, or atmospheric pressure. A person skilled in the art appreciates that freezing can be performed at different speeds to control the size of the ice crystals formed. A person skilled in the art appreciates that slow freezing or annealing may produce a frozen product with large ice crystals, whilst rapid freezing may produce a frozen product with smaller crystals.

In embodiments, the entire lyophilization method may be performed in a lyophiliser or freeze dryer, such as a manifold freeze-dryer, a rotary freeze-dryer or a tray style freeze-dryer, preferably a FreeZone 1 freeze-dryer (Labconco, USA). In embodiments the product is first frozen, and the primary and secondary drying steps performed in a lyophiliser or freeze dryer, such as a manifold freeze-dryer, a rotary freeze-dryer or a tray style freeze-dryer, preferably a FreeZone 1 freeze-dryer (Labconco, USA). In embodiments the total lyophilization process may be performed over 5 hours to 100 hours, 5 hours to 72 hours, 10 hours to 72 hours, 15 hours to 72 hours, 12 hours to 48 hours, 12 hours to 36 hours, 18 hours to 30 hours, 20 hours to 30 hours, or 20 hours to 28 hours.

The lyophilized product may be stored at any temperature. In embodiments, the lyophilized product may be stored at a maximum of 23° C., 25° C., 27° C., 30° C., 35° C., 40° C., 45° C., 50° C., 60° C., 70° C.

As used herein, the term ‘lyophilized’ refers to a product that has undergone the lyophilization process, i.e., dehydrated at a low temperature. Lyophilized products have enhanced stability compared to non-lyophilized products and therefore tend to have extended storage life compared to non-lyophilized products. In addition, lyophilized products have a lower risk of microbial contamination, oxidation, loss of volatile constituents; Lyophilized products may be reconstituted in liquids, such as water. In embodiments the conditioned culture medium is lyophilized. In embodiments where the conditioned culture medium is lyophilized, the lyophilized conditioned culture medium may be reconstituted in hyaluronic acid at concentrations described herein. In embodiments where the conditioned culture medium is lyophilized, it may be mixed with one or more other lyophilized agents, such as lyophilized hyaluronic acid. The mixture of lyophilized conditioned culture medium and one or more other lyophilized agents may then be reconstituted in a liquid, such as water. In embodiments a composition comprising, consisting, or consisting essentially of the conditioned culture medium and hyaluronic acid is lyophilized. In embodiments, the lyophilized composition of conditioned culture medium and hyaluronic acid may be used as a ‘sponge’ or may be reconstituted in a liquid, cream, or gel before use, such as water, glycerin, oil, alcohol, or a silicone-containing compound.

The invention also provides methods of producing the composition described herein, said method comprising a) culturing MSCs, and b) collecting conditioned culture medium therefrom. In embodiments, the method further comprises c) mixing the conditioned culture medium with at least one other agent, preferably hyaluronic acid. In embodiments the method comprises a step before a) comprising isolating the MSCs, preferably isolating hMSCs from an umbilical cord, more preferably isolating hWJ-MSCs from Wharton's jelly. In embodiments the isolation of MSCs is performed using methods described herein. In embodiments a) of culturing MSCs is performed using methods described herein. In embodiments, the culture medium a) and/or b) is any suitable culture medium, preferably any suitable culture medium described herein, preferably DMEM. In embodiments the medium of a) and/or b) may be supplemented with serum, preferably fetal bovine serum, preferably at a concentration between 0.1% to 30%, 1% to 30%, 1% to 25%, 1% to 20%, 1% to 15%, 5% to 20%, 5% to 15% or 8% to 12% serum (v/v). The MSCs are then cultured in a) using methods described herein until they reach between 40% and 100% confluency, preferably between 40% and 90%, 50% and 80%, or 60% and 90%, more preferably between 80% and 90%. In embodiments a) further comprises passaging MSCs. In embodiments, passaging is performed using methods described herein. In embodiments, passaging in a) comprises counting the cell numbers using methods described herein and plating 100 to 1,000,000 MSCs, preferably 1000 to 500,000 MSCs, more preferably, 1000 to 250,000 MSCs, or about 170,000 MSCs into a fresh culture vessel using methods described herein. In embodiments, a) further comprises replacing the culture medium with fresh culture medium, preferably a serum free and/or phenol red-free medium. In embodiments, the MSCs are cultured in the fresh culture medium for between 2 to 100 hours, 2 to 72 hours, 5 to 100 hours, 5 to 72 hours, 2 to 60 hours, 5 to 60 hours, 2 to 50 hours, 5 to 50 hours, 10 to 100 hours, 10 to 72 hours, 10 to 60 hours, 10 to 50 hours, 15 to 100 hours, 15 to 72 hours, 15 to 60 hours, 15 to 50 hours, 20 to 100 hours, 20 to 72 hours, 20 to 60 hours, 20 to 50 hours, 24 to 72 hours, 24 to 60 hours, 24 to 50 hours, 24 to 48 hours, 48 to 72 hours, 30 to 70 hours, 38 to 58 hours, 40 to 56 hours, or 40 to 50 hours. In embodiments, b) of collecting the conditioned medium is performed using the methods described herein. In embodiments, b) comprises transferring the culture medium from the cell culture vessel into a suitable container. In embodiments, a step between b) and c) may be performed comprising storing the conditioned culture medium can be stored at temperatures between 10° C. to −130° C., preferably 4° C. to −120° C., more preferably −20° C. to −120° C., even more preferably between −80° C. to −120° C. In embodiments the method comprises an addition step in c) of lyophilizing the conditioned culture medium. In these embodiments, the lyophilized is mixed with a second agent, preferably hyaluronic acid c). In embodiments, the hyaluronic acid may also be lyophilized c). In embodiments, step c) comprises mixing at least 5%, 10%, 15% or 20% conditioned culture medium with at least a second agent, preferably hyaluronic acid (v/v). In embodiments, the final concentration of hyaluronic acid in the mixture of c) is between 0.5 mg/ml to 100 mg/mL, 0.5 mg/mL to 90 mg/mL, 0.5 mg/mL to 80 mg/mL, 0.5 mg/mL to 70 mg/mL, 0.5 mg/mL to 60 mg/mL, 0.5 mg/mL to 50 mg/mL, 0.5 mg/mL to 40 mg/mL, 0.5 mg/mL to 30 mg/mL, 0.5 mg/mL to 25 mg/mL, 0.5 mg/mL to 20 mg/mL, 0.5 mg/mL to 15 mg/mL, 0.5 mg/mL to 10 mg/mL, 0.5 mg/mL to 5 mg/mL, 1 mg/mL to 100 mg/mL, 1 mg/mL to 80 mg/mL, 1 mg/mL to 60 mg/mL, 1 mg/mL to 50 mg/mL, 1 mg/mL to 40 mg/mL, 1 mg/mL to 30 mg/mL, 1 mg/mL to 25 mg/mL, 1 mg/mL to 20 mg/mL, 1 mg/mL to 15 mg/mL, 1 mg/mL to 10 mg/mL, 1 mg/mL to 5 mg/mL, 1 mg/mL to 4 mg/mL, 1 mg/mL to 3 mg/mL, 0.5 mg/mL to 3 mg/mL or 1 mg/mL to 2 mg/mL. In embodiments, the method further comprises d) lyophilizing the mixture of conditioned culture medium and hyaluronic acid. In embodiments, the lyophilization d) is performed as described herein.

### Treatment/Prevention

As used herein, the terms ‘subject’, ‘patient’ or ‘individual’ are used interchangeably and refer to vertebrates, preferably mammals, such as humans and non-human primates, as well as other animals such as bovine, equine, canine, ovine, feline, murine and the like. In preferred embodiments, the subject, patient, or individual is human. Accordingly, the term ‘subject’, ‘patient’ or ‘individual’ as used herein means any mammal diagnosed with, predisposed to, or suspected of having a disorder, disease, or condition. In preferred embodiments, the ‘subject’, ‘patient’ or ‘individual’ as used herein is diagnosed with, predisposed to, or suspected of having a disorder, disease, or condition of the skin (i.e., a skin disorder, skin disease or skin condition) and/or a disorder, disease or condition associated with tortuous blood vessels and/or a disorder, disease or condition associated with an unregulated angiogenic process.

As used herein, the terms ‘disorder’, ‘disease’ or ‘condition’ are used interchangeably to refer to the presence of pathology and/or the presence of clinical signs and symptoms. In embodiments, the disorder, disease, or condition is of the skin (i.e., a skin disease, disorder, or condition). In embodiments the disorder, disease or condition is associated with tortuous blood vessels. In embodiments, the disorder, disease, or condition is a skin disorder, disease or condition associated with tortuous blood vessels. As used herein, ‘associated with tortuous blood vessels’ means any disease, disorder or condition that is characterized by the presence of tortuous blood vessels or generates tortuous blood vessels. In embodiments, the disease, disorder, or condition is selected from varicose veins, psoriasis, hemorrhoids, rosacea, acne, crystal skin (calcinosis cutis) and dermatitis. In embodiments the dermatitis is atopic dermatitis or stasis dermatitis (varicose eczema). In embodiments the psoriasis is psoriasis vulgaris.

As used herein, ‘vessels’ or ‘blood vessels’ are used interchangeably to refer to the tubular structures through which blood circulates. As used herein, ‘tortuous blood vessels’ or ‘tortuous vessels’ are used interchangeably to refer to blood vessels with an abnormal morphology compared to a control blood vessel. Blood vessels tend to be relatively straight, however tortuous blood vessels are characterized by additional twists and turns in the blood vessels, such as curving, angulation/kinking, spiral twisting, or looping (Han, 2012. Twisted blood vessels: symptoms, etiology, and biomechanical mechanisms. J Vasc Res, 49(3): 185-197). Tortuous blood vessels can be any blood vessel in a subject, such as arteries, veins, arterioles, venules, and/or capillaries. In embodiments, the tortuous blood vessels are capillaries. In embodiments, the tortuous blood vessels are located in the skin, i.e., the epidermis, dermis, or hypodermis.

Moderate-severe tortuous blood vessels in the skin may be visualized by eyesight alone. Alternatively, the skilled person may use one or more standard imaging techniques in the art for visualizing blood vessels in the skin, such as magnifiers (e.g., dermoscopes), photography (with varying magnifications such as ×0.8, ×1.6, ×2, ×4, ×6 or ×10 magnification), infrared imaging, near-infrared imaging (such as those described by Khan et al. 2021, Visualization of superficial vein dynamics in dorsal hand by near-infrared imaging in response to elevated local temperature. J of Biomedical Optics, 26(2), 026003), capillaroscopy, Optoacoustic imaging (OAI), raster-scanning optoacoustic mesoscopy (RSOM) (as described in Nitkunanantharajah et al., 2020. Three-dimensional optoacoustic imaging of nailfold capillaries in systemic sclerosis and its potential for disease differentiation using deep learning. Scientific Reports, 10:16444), tourniquet tests (such as the Trendelenberg test), transillumination, computed tomography (including optical coherence tomography (OCT) as described in Holmes et al., 2019. Dynamic Optical Coherence Tomography Is a New Technique for Imaging Skin Around Lower Extremity Wounds. The International Journal of Lower Extremity Wounds, 18(1): 65-74), ultrasonography (including handheld Doppler, duplex or ultrasound methods as described in WO 2018/222724, WO 2020/252463), or sonography. The skilled person also appreciates that various liquids or gels can be used to improve visualisation of imaging methods such as alcohol, immersion oil, water, or ultrasound gels (Ayhan et al, 2015. Vascular structures in dermascopy. An Bras Dematol, 90(4): 545-553).

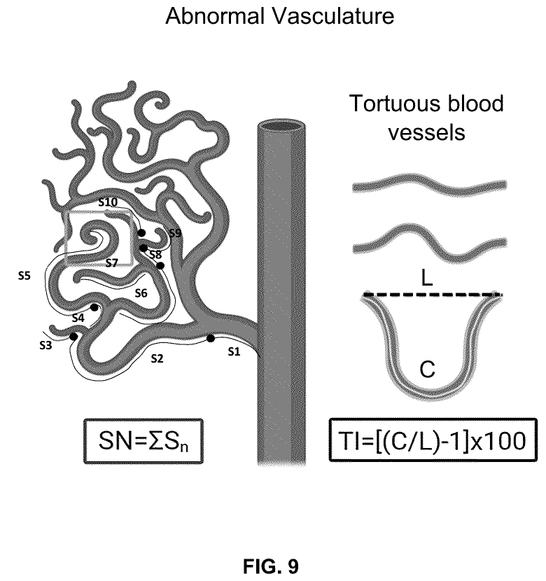

The skilled person appreciates that upon visualization, different methods may be used to quantify the presence of tortuous blood vessels in a subject, and/or the severity of the tortuous blood vessels. For example, a skilled person may measure the absolute number of tortuous blood vessels as compared to a control (or reference) number in healthy subjects, the ratio of tortuous blood vessels to normal blood vessels, the number of total segments (or ‘branches) in the blood vessel(s), the ratio of the curve length over the shortest line connecting the start and end of the vessel (referred to as the tortuosity index, calculated as shown in FIG. 9 and as described in Virgilio et al., 2017. Vertebral Tortuosity Index in Patients with Non-Connective Tissue Disorder-Related Aneurysm Disease. European Journal of Vascular and Endovascular Surgery, 53(3): 425-430), total curvature of the blood vessel, mean curvature of the blood vessels, cumulative sum of the angle between segment vectors normalized by vessel length, or ratio of deflection amplitude versus the wavelength (as described in Han et al., 2012, Twisted blood vessels: symptoms, etiology, and biomechanical mechanisms. J Vasc Res, 49(3): 185-197).

As used herein, ‘control’ refers to comparison to a control blood vessel in a healthy subject, or population.

The terms ‘treating’, ‘treat’, ‘treated’ or ‘treatment’ as used herein refer to reducing the severity and/or frequency of symptoms, reducing the underlying pathological markers, eliminating symptoms and/or pathology, arresting the development or progression of symptoms and/or pathology, slowing the progression of symptoms and/or pathology, eliminating the symptoms and/or pathology, or improving or ameliorating pathology/damage already caused by the disease, condition, or disorder. In embodiments, treatment refers to at least the reduction in the pathology of tortuous blood vessels, such as a reduction in the number of tortuous blood vessels, or a reduction in the tortuosity of blood vessels. In embodiments, treatment refers to at least the reduction of one or more of: the occurrence or severity of erythema, reduced epidermal thickness, reduced number or severity of scales, spots or lesions, reduced area or severity of dry or cracked skin, and reduced occurrences or severity of itching, burning or discomfort.

The terms ‘preventing’ or ‘prophylaxis’ as used herein refer to the prevention of the occurrence or recurrence of symptoms and/or pathology, delaying the onset of symptoms and/or pathology, or preventing progression of symptoms and/or pathology. Therefore, ‘preventing’ or ‘prophylaxis’, applies when a subject has, is suspected of having, or is at risk of a disorder, disease, or condition, particularly a skin disorder, disease, or condition, and/or a condition associated with tortuous blood vessels. In embodiments the subject may not yet display symptoms or pathology. In embodiments, the symptoms or pathology prevented include at least one or more of: increased number of tortuous blood vessels, increased tortuosity of blood vessels, increased blood vessel diameter, increased occurrence of erythema, increased severity of erythema, increased epidermal thickness, increased number or severity of scales, spots or lesions, increased area, or severity of dry or cracked skin, and increased occurrences or severity of itching, burning or discomfort.

As used herein, ‘treatment or prevention’ is referring to the use of the disclosed pharmaceutical composition or method to treat or prevent a disease, disorder, or condition in a subject, preferably a skin disease, disorder, or condition and/or a disease disorder or condition associated with tortuous blood vessels.

In another embodiment, the production method of pharmaceutical compositions comprising HA and conditioned medium includes the following stages:
1) Dissolve 26.4 mg OF HA in 13.2 mL of Milli-Q water at room temperature, generate a 2 mg/ml solution of HA in Milli-Q water at room temperature;
2) Mix the solution obtained in step 1) with conditioned medium to give a final solution with a concentration of 20% v/v of conditioned medium.
3) Freeze samples at −20° C. for 24 hours;
4) Lyophilize the mixture in a lyophilizer at 0.02 mbar for 24 hours using a condenser temperature of −54° C.; and
5) Store lyophilized sponges at room temperature until use.

### EXAMPLES

### Example 1: Preparation of the Pharmaceutical Composition Comprising the hWJ-MSC Cell Secretome and Hyaluronic Acid

hWJ-MSC Cell Collection

The hWJ-MSC cells were isolated from umbilical cords obtained from normal term pregnancies obtained by informed consent. The analysis of the samples was approved by the Ethics committee of the University of Chile and Dr. Luis Tisné Brousse Hospital.

The cultures were generated and expanded from fresh umbilical cords that were processed no more than one day after arrival in the laboratory, following standard isolation protocols previously described (Edwards, S, et al. 2014. Functional analysis reveals angiogenic potential of human mesenchymal stem cells from Wharton's jelly in dermal regeneration. Angiogenesis, 17(4), 851-866; and Prieto, C., et al. 2017. Netrin-1 acts as a non-canonical angiogenic factor produced by human Wharton's jelly mesenchymal stem cells (WJ-MSC). Stem Cell Research and Therapy, 8(1), 1-15). All primary hWJ-MSC cultures were used between passages 2-8.

hWJ-MSC were sowed in culture plates of 60 mm diameter (P60), considering between 80-90% confluence, in DMEM medium supplemented with 10% SFB and antibiotics (100 U/mL penicillin/streptomycin. The cultures were kept in a humid atmosphere containing 5% CO₂ at 37° C.

hWJCM Collection

The cell density was evaluated under a microscope so that 170,000 cells were sowed per plaque P60. hWJ-MSC cultures were grown to 80-90% confluence, were cultured in DMEM without red phenol (PRF) for 48 hours, and hWJCM were collected fresh, immediately frozen in liquid nitrogen, and stored at −80° C. until use. At least three different hWJCM, obtained from different hWJ-MSC cultures, were mixed for experimental trials.

Preparation of Control Composition Containing Hyaluronic Acid and Compositions Containing hWJCM with Hyaluronic Acid

Hyaluronic acid (HA, MW˜330 kDa) was acquired from Inquiaroma (Barcelona, Spain) and used according to the manufacturer's instructions. Milli-Q water was used for testing. To prepare formulations with sponge consistency, an initial solution comprising 1.6 mg/ml HA (MW˜330 kDa) and hWJCM was prepared. The initial solution was obtained by dissolving 26.4 mg of HA in 13.2 mL of Milli-Q water at room temperature, then this solution was mixed with 3.3 mL of conditioned medium to give a final volume of 16.5 mL. The resulting mixture (HA hWJCM) was transferred in 96-well plates (100 μl/well). The samples were frozen at −20° C. for 24 hours and lyophilized in a FreeZone 1 freeze dryer (Labconco, USA). Sublimation was performed at 0.02 mbar for 24 hours (condenser temperature was −54° C.) and HA hWJCM sponges were obtained which were stored at room temperature. Hyaluronic acid control sponges (HA Ctrl), without hWJCM, were obtained following the same process.

### Example 2: HA hWJCM has Angiogenic Potential in an In Vivo CAM Model

An assessment of the angiogenic potential of the pharmaceutical composition was carried out to determine whether this property was maintained after the lyophilization process in a chorioalantoid membrane (CAM) assay of chicken embryo. For this test, fertilized chicken eggs (Rock iso, Agricola Chorombo, Chile) were incubated at 38.5° C. with constant humidity at 75%. On development day 1 (E1), 2 mL of albumin were extracted from each egg and a round window (2 cm²) was created in E4. On day E8, the CAM vasculature was photographed; subsequently, each experimental condition was placed on top of the CAM and photographed; for each condition, 10 eggs were used. The CAM trial included testing 20 μl of the following conditions: PRF DMEM (negative control), VEGF 50 ng/ml (positive control) and hWJCM. Additionally, sponges (5 mm diameter) of HA Ctrl or HA hWJCM were evaluated. The stimulus was positioned between the intersection of two large blood vessels and placed on a 12 mm coverslip. The results of the stimulation were evaluated in E12 by photographs of the CAM area on which the coverslip was placed, using a magnifying glass of 0.8 and a digital camera HD IC80 (Leica, Heidelberg, Germany). To improve the visualization of the vessels, 3 mL of a 1:1 cream and distilled water solution were injected under the CAM before photographing each egg. For quantification, all blood vessels that intercepted the coverslip edges and the total number of vessels within a 6 mm radius of the sponges were considered in E12. The results were normalized according to the number of vessels in E8.

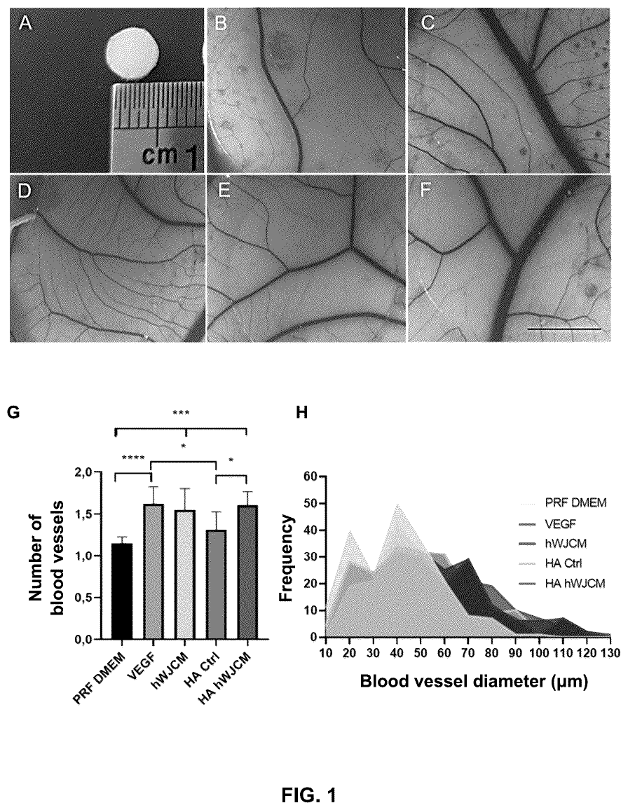

After 4 days of CAM stimulation, it was possible to observe an increase in blood vessels under conditions of hyaluronic acid alone (HA Ctrl), VEGF, Wharton gelatin-derived cell secrete (hWJCM) and pharmaceutical composition comprising secretome plus hyaluronic acid (HA hWJCM) compared to DMEM PRF control condition as shown in FIGS. 1A-H. The results obtained after quantification, shown in FIG. 1G, show significant differences between the membranes stimulated with PRF DMEM and those with hWJCM and HA hWJCM. In these conditions, a number of total vessels was obtained similar to that of VEGF positive control, suggesting a strong angiogenic potential by these stimuli. Although HA Ctrl has a greater number of blood vessels than PRF DMEM, no significant differences were found between the above conditions. In addition to the above, FIG. 1H shows that vessels stimulated with VEGF, hWJCM, HA Ctrl and HA hWJCM were found to have a larger diameter compared to the PRF DMEM condition; no significant differences were observed between these stimuli.

### Example 3: Evaluation of the Therapeutic Potential of hWJCM and HA hWJCM in a Psoriatic-Like Dermatitis (PD) Model

Having confirmed that HA hWJCM maintains the angiogenic properties, and therefore the potential functions of the secretome of the hWJ-MSC, the next objective was to establish whether the pharmaceutical composition HA hWJCM attenuates the signs of IMQ-induced PD in BALB/c mice.

For this purpose, BALB/c mice of 8 to 12 weeks of age of both sexes, and males C57BL/6-IL17A were obtained from the Bioterium of the University of Chile and were maintained under strict specific conditions free of pathogens at 25° C.±2° C. and relative humidity of 45%, with a cycle of 12 hours of light/dark. The mice had access to food and water ad libitum. All experiments adhered to the principles of the Helsinki Declaration and were approved by the institutional and bioethical committees of the University of Chile. The mice were divided into groups according to the experimental design. The control group (Ctrl) corresponds to mice that were shaved or shaved mice that received Vaseline, the IMQ vehicle. Mice in all remaining groups received 62.5 mg as a topical daily dose of commercially available Imiquimod (IMQ; 5%, Labimiq ITF-labeled) on the depilated back for either 7 or 12 consecutive days to establish an IMQ-induced PD model.

The experimental treatments evaluated in a first set of experiments correspond to: DMEM (hWJCM vehicle) or subcutaneous hWJCM injections (100 μl distributed at 5 locations on the back of the mice), clobetasol propionate 0.05% (Opko Lab.) (Clob) sufficient to cover the back of the animals. A second set of experiments also included HA Ctrl (five 5 mm sponges per mouse), and HA hWJCM sponges (five 5 mm sponges per mouse). At least three animals per group were considered. During the induction period, the PASI index was calculated as well as the percentage weight variation of the animals. Prior to and during the experimental period, all mice were evaluated to measure their overall health status. After completing the 7 or 12-day PD induction protocol, plus an additional 24 hours, the mice were euthanized by isoflurane overdose.

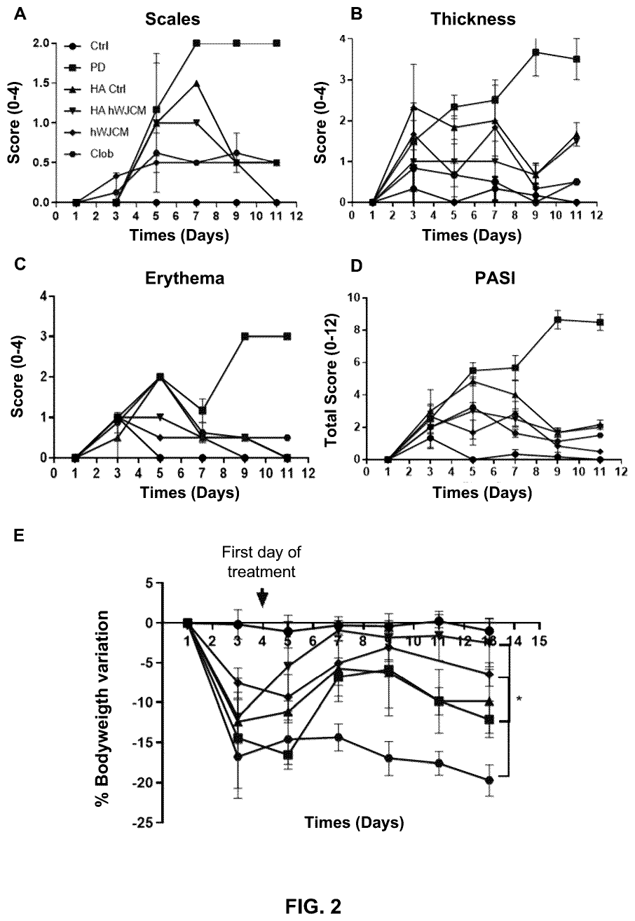

FIGS. 2A-D show the evolution of the different parameters contributing to the PASI index over 11 days. On the last day, the condition with the highest PASI total score was PD (8.5±0.5), followed by those treated with HA Ctrl (2.17±0.29), HA hWJCM (2±0), Clob (1.5±0), hWJCM (0.5±0), and finally Ctrl (0±0). Interestingly as to the weight variation of the animals compared to the first day, it was found that after the first application of IMQ, they lose weight. This result is expected, and coincides with literature reports, since IMQ induces strong dermal inflammation and sores with the consequent discomfort of the animals, which affects their feeding capacity. However, once PD treatment was started, the mice given hWJCM started to gain weight and on the last day of treatment, these individuals had a near total recovery (Δ=−2.51%), reaching a value close to that of the Ctrl condition (Δ=−0.99%) Result that could not be seen under the other experimental conditions. By evaluating the same weight parameter, the hWJCM-treated mice (Δ=−6.47%) follow treatment with HA hWJCM; mice treated with HA Ctrl decreased their weight to a level close to that of PD mice (−9.84% and −12.11% respectively), shown in FIG. 2E.

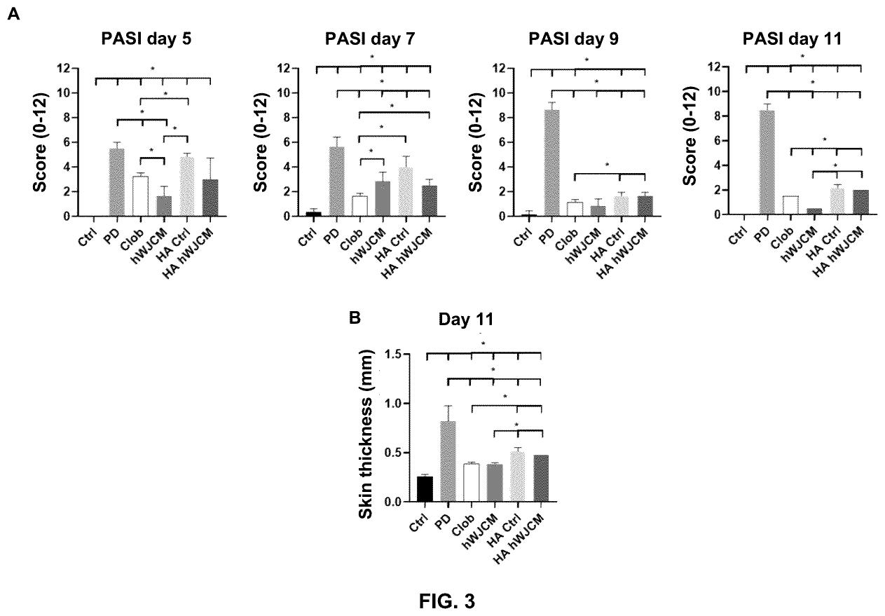

With the results obtained, it was possible to conclude that after only 1 days of treatment (corresponding to day 4 of induction of psoriasiform dermatitis), topical administration of Clob and subcutaneous injection of hWJCM resulted in a drastic decrease in PASI index compared to HA Ctrl and HA hWJCM. However, after 3 days of treatment (corresponding to day 7 of induction of psoriasiform dermatitis), it was possible to observe a significant difference in the reduction of the PASI index given the treatments HA Ctrl and HA hWJCM, the latter being the most effective (FIG. 3A). In relation to the skin thickness, it was seen that the treatment of PD caused a significant decrease in the skin, shown in FIG. 3B.

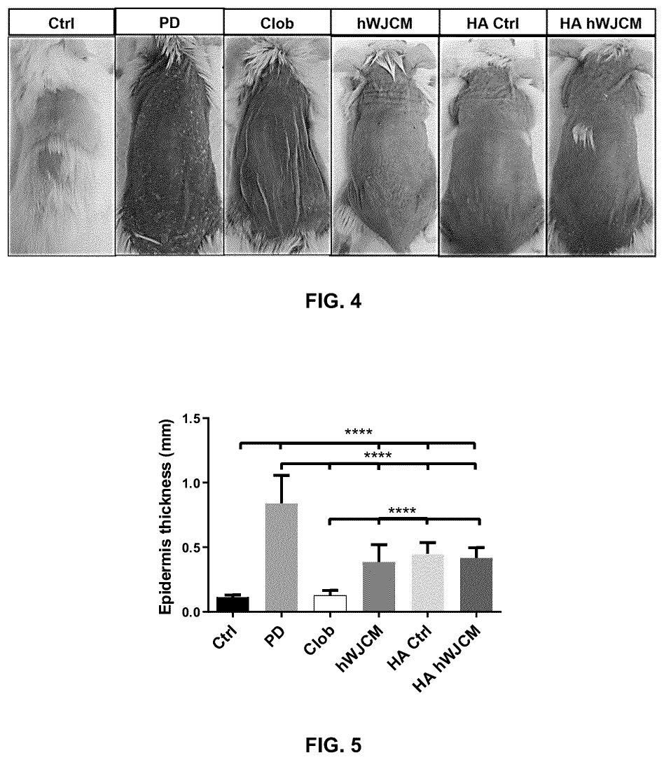

The high PASI score for PD animals is given by a high degree of erythema, scaling, and thickening of the skin. On the other hand, the decrease in this index is due to phenotypic changes in the dorsal skin of mice induced with PD against the treatments hWJCM, Clob, HA Ctrl, and HA hWJCM as shown in FIG. 4, a reduction in the PASI index was achieved by 94.12%, 82.35%, 74.51% and 76.47% respectively. In these experiments, acanthosis, parakeratosis, elongation of the epidermal peaks and cellular infiltrate were clearly observed in the condition of PD. Treatment with Clob, hWJCM, HA Ctrl and HA hWJCM induced a decrease in epidermal hyperplasia and parakeratosis. In addition, with regard to cellular infiltrating the dermis, a significant decrease was found only when comparing Clob and HA hWJCM treatments. The PD condition showed the greatest thickness of the epidermis followed by hWJCM, HA Ctrl, HA hWJCM and Clob (FIG. 5).

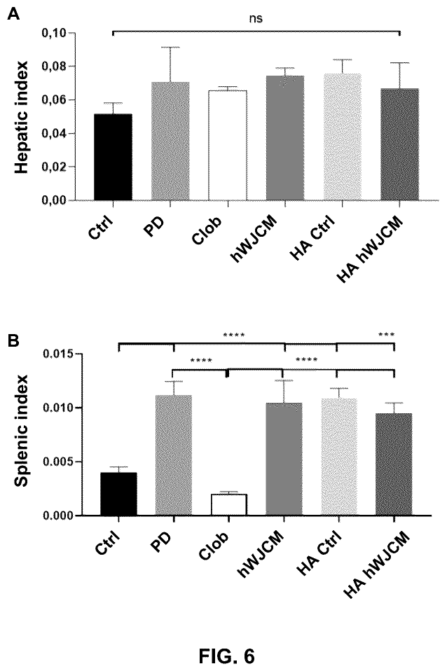

After the euthanasia of the individuals, carried out on day 13, the spleen and liver were removed from each animal. Once this has been done, there was no change in the appearance of livers of PD-induced conditions with respect to Ctrl, eventually obtaining fairly similar liver index values for all experimental conditions. Quite the contrary, it was possible to show an increase in the size of the spleen of those individuals induced with PD, both in length and in width, which resulted in a higher splenic index for these animals, These were found to be significantly greater than the one obtained in condition Ctrl. The above is shown in FIG. 6A and FIG. 6B respectively.

### Example 4: The Pharmaceutical Composition of the Invention Decreases the Amount of Tortuous Blood Vessels in a PD Model

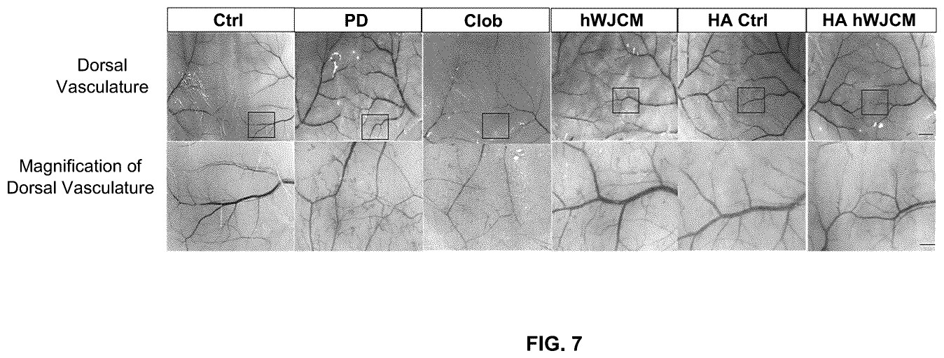

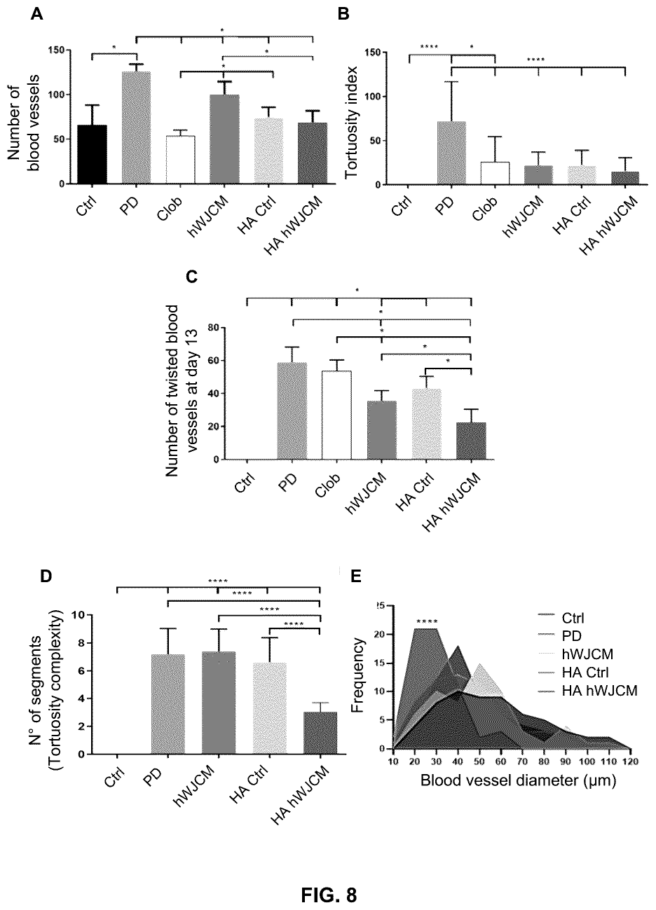

Angiogenesis consists of the growth of new blood vessels from pre-existing vessels, which is essential during the development and maintenance of psoriasis. The morphological changes described in the blood vessels of psoriatic patients suggest the presence of unregulated angiogenesis resulting in aberrant vasculature formation. To assess these microcirculatory alterations, prior to obtaining skin cuts for histological analysis, images of the skin of the back were acquired to observe and analyze in greater detail the architecture of the blood vessels (FIG. 7). The number of blood vessels decreased with all topical treatments (FIG. 8A). Surprisingly, topical treatment of PD mice with HA Ctrl and HA hWJCM drastically reduced the tortuosity rate, similar to that found after hWJCM injection or Clob treatment (FIG. 8B). With regard to the number of twisted blood vessels, the most notable difference was between PD mice and mice treated with HA hWJCM. Mice treated with Clob and PD showed a similar number of tortuous blood vessels at the end of treatment (FIG. 8C). It should be noted that after analyzing the complexity of the tortuosity of the tortuous blood vessels as the total number of segments for each vessel, we observed that the vasculature of the mice treated with HA hWJCM was less complex than that observed under conditions PD, hWJCM and HA Ctrl (FIG. 8D). Finally, the smaller diameter of the new vessels produced as a result of PD induction returned to normal after concomitant treatments (FIG. 8E). Taken together, these findings show that HA hWJCM was the only treatment that improved the structure of PD blood vessels by multiple criteria.

It is important to note that the decrease in the quantity of tortuous blood vessels caused by the HA hWJCM treatment is unexpected and surprising, as this treatment demonstrates a synergistic effect between the evaluated secretome and hyaluronic acid.

### INDUSTRIAL APPLICATION

The present invention has a great application in the pharmaceutical and biomedical industry. Thus, the present invention provides a new product to treat alterations in blood vessels in different diseases.

1. A pharmaceutical composition comprising a lyophilized conditioned culture medium of human mesenchymal stem cells and a polymeric matrix of hyaluronic acid.

2. The pharmaceutical composition of claim 1, wherein the human mesenchymal stem cells are obtained from Wharton's gelatin.

3. A pharmaceutical composition comprising a combination of trophic factors expressed by human mesenchymal stem cells and a polymeric matrix of hyaluronic acid.

4. The pharmaceutical composition of claim 3, wherein the pharmaceutical composition is lyophilized.

5. The pharmaceutical composition of any preceding claim, wherein the composition comprises at least one, two, three, four, five, ten, twenty, thirty or more trophic factors selected from: Angiogenin, Ang-1, Ang-2, amphiregulin, artemin, Coagulation factor 3, CXCL16, DPPIV, EGF, EG-VEGF, Endoglin, endothelin-1, aFGF, bFGF, FGF-4, FGF-7, GDNF, GM-CSF, HB-EGF, HGF, IGFBP-2, IGFBP-3, IL-1B, TGF-B1, LEPTIN, MCP-1, MMP-8, MMP-9, NRG1-B1, PD-ECGF, PDGF-AA, PDGF-AB, PDGF-BB, PERSEFIN, PGF, Prolactin, UPA, VEGF, VEGF-C, activin A, angiostatin, HIF-1, Endostatin/CollagenXVIII, IGFBP-1, MIP1a, PTX3, PF4, Serpin B5, Serpin E1, Serpin F1, TIMP-1, TIMP-4, TSP-1, TSP-2, vasoinhibin, ADAMTS-1, and Netrins.

6. The pharmaceutical composition of any preceding claim, wherein the composition comprises a combination of trophic factors including Angiogenin, Ang-1, Ang-2, amphiregulin, artemin, Coagulation factor 3, CXCL16, DPPIV, EGF, EG-VEGF, Endoglin, endothelin-1, aFGF, bFGF, FGF-4, FGF-7, GDNF, GM-CSF, HB-EGF, HGF, IGFBP-2, IGFBP-3, IL-1B, TGF-B1, LEPTIN, MCP-1, MMP-8, MMP-9, NRG1-B1, PD-ECGF, PDGF-AA, PDGF-AB, PDGF-BB, PERSEFIN, PGF, Prolactin, UPA, VEGF, VEGF-C, HIF-1, activin A and/or angiostatin.

7. The pharmaceutical composition of any preceding claim, wherein the composition comprises a combination of trophic factors including Angiostatin, Endostatin/CollagenXVIII, IGFBP-1, MIP1a, PTX3, PF4, Serpin B5, Serpin E1, Serpin F1, TIMP-1, TIMP-4, TSP-1, TSP-2, vasoinhibin, ADAMTS-1, HGF, Netrins and bFGF.

8. The pharmaceutical composition of any preceding claim, wherein the hyaluronic acid is at a concentration between 0.5 mg/ml and 3 mg/ml, preferably between 1 mg/ml and 2 mg/ml.

9. Use of the pharmaceutical composition according to any of claims 1 to 8, for the manufacture of a medicinal product for preventing and/or treating a disease, disorder, and/or condition that generates tortuous blood vessels.

10. The use of claim 9, wherein the tortuous blood vessels are generated by a skin disease, disorder, and/or condition.

11. The use of claim 10, wherein the skin disease, disorder, and/or condition is selected from: varicose veins, psoriasis, hemorrhoids, rosacea, acne, crystal skin and atopic dermatitis.

12. The use of claim 11, wherein psoriasis is psoriasis vulgaris.

13. A pharmaceutical composition according to any of claims 1 to 8, for use in preventing and/or treating a disease, disorder, and/or condition that generates tortuous blood vessels.

14. The pharmaceutical composition of claim 13, wherein the disease, disorder, and/or condition is a skin condition, preferably wherein the skin condition is selected from: varicose veins, psoriasis, hemorrhoids, rosacea, acne, crystal skin and atopic dermatitis.

15. A method of preventing or treating a skin disease, disorder and/or condition in a subject in need thereof, the method comprising administering to the subject a pharmaceutically effective amount of the pharmaceutical composition according to any of claims 1 to 8.

16. The method according to claim 15, wherein the disease, disorder, and/or condition is a skin condition.

17. The method according to claim 16, wherein the skin condition is selected from: varicose veins, psoriasis, hemorrhoids, rosacea, acne, crystal skin and atopic dermatitis.

18. A method of producing the pharmaceutical composition of any of claims 1 to 8, said method comprising:
(a) mixing human mesenchymal stem cells with a culture medium;
(b) culturing the human mesenchymal stem cells in the culture medium for obtaining a conditioned culture medium;
(c) collecting the conditioned culture medium obtained; and
(d) mixing the collected conditioned culture medium with hyaluronic acid for obtaining the pharmaceutical composition.

19. The method of claim 18, further comprising a step of lyophilizing the pharmaceutical composition.

20. The method of claim 18, further comprising a step of isolating human mesenchymal stem cells from Wharton's jelly, before step (a).

21. The method according to any of claims 18 to 20, wherein the culture medium comprises Dulbecco's Modified Eagle Medium (DMEM).

22. The method according to claims 18 to 21, wherein the culture medium comprises fetal bovine serum.

23. The method of claim 22, wherein the concentration of fetal bovine serum is between 0.1% to 30%, more preferably between 5% and 15%.

24. The method according to any of claims 18 to 23, wherein the human mesenchymal stem cells in step (a) are mixed with the culture medium in vessels, mixing between 100 to 1,000,000 cells per vessel, more preferably between 1,000 and 250,000 cells per vessel.

25. The method according to any of claims 18 to 24, wherein the cells in step (b) are cultured in an incubation temperature between 20° C. and 40° C., preferably between 30° C. and 37° C.

26. The method according to any of claims 18 to 25, wherein the human mesenchymal stem cells are cultured until a confluence value of between 50% and 100%, preferably between 70% and 90%, more preferably between 80% and 90%, is achieved.

27. The method according to any of claims 18 to 26, further comprising in step (b) a step of replacing the culture medium, preferably after a confluence value of between 50% and 100% is achieved, with a second culture medium comprising Dulbecco's Modified Eagle Medium (DMEM) without phenol red.

28. The method according to any of claims 18 to 27, wherein the step of collecting the conditioned culture medium is done 12-72 hours after mixing the human mesenchymal stem cells with the culture medium.

29. The method of claim 27, wherein the step of collecting the conditioned culture medium is done after 48-72 hours incubation in the second culture medium.

30. The method according to any of claims 18 to 29, wherein the final concentration of hyaluronic acid in step (d) is between 0.5 to 3 mg/mL, preferably 1 to 2 mg/mL.

31. The method according to any of claims 18 to 30, further comprising after step (c), storing the conditioned culture medium at −80° C. until lyophilization and/or use.

32. The method according to any of claims 18 to 31, further comprising after step (c), lyophilizing the collected conditioned culture medium.

33. The method according to any of claims 18 to 32, wherein the steps (a), (b), and (c) comprise the steps of:
(a) preparing Petri dishes with a culture medium of DMEM medium supplemented with 10% Fetal Bovine Serum (FBS);
(b) adding about 170,000 cells of human mesenchymal stem cells from Wharton's jelly in said Petri dishes;
(c) culturing the cells at 37° C. until they reach a confluence of 80-90%;
(d) replacing the culture medium with a second culture medium of DMEM medium without phenol red (SRF);
(e) collecting a conditioned culture medium after culturing the cells for 48 hours in the second culture medium; and
(f) storing the conditioned culture medium at −80° C. until use.

34. The method according to any of claims 18 to 33, wherein the step (d) comprises the steps of:
a) generating a solution of 2 mg/ml of hyaluronic acid in Milli-Q water at room temperature;
b) mixing the hyaluronic acid solution with the conditioned culture medium collected in any of claims 18 to 33, for obtaining a final solution with a concentration of 20% v/v of conditioned culture medium;
c) transferring the final solution to culture plates;
d) freezing the culture plates containing the final solution at −20° C. for 24 hours;
e) lyophilizing the frozen final solution at 0.02 mbar for 24 hours using a condenser temperature of −54° C. for obtaining a pharmaceutical composition; and
f) storing the lyophilized pharmaceutical composition at room temperature until use.

35. A pharmaceutical composition obtained by a method according to any of claims 18 to 34.
